(19) United States
(12) Patent Application Publication
(10) Pub. No.: US 2006/0140933 A1
(43) Pub. Date: Jun. 29, 2006

(54) Compositions and methods for treating rage-associated disorders

(75) Inventors: Debra D. Pittman, Windham, NH (US); Brian Clancy, Ashland, MA (US); Glenn Larsen, Sudbury, MA (US); William L. Trepicchio, Andover, MA (US); Fionula Mary Brennan, Middlesex (GB); Marc Feldmann, London (GB); Brian John Maurice Foxwell, Middlesex (GB); Jeffrey L. Feldman, Arlington, MA (US)

Correspondence Address: FISH & NEAVE IP GROUP;ROPES & GRAY LLP, ONE INTERNATIONAL PLACE, BOSTON, MA, 02110-2624, US

(73) Assignee: Wyeth and Imperial College Innovations Limited, London (GB)

(21) Appl. No.: 10/643,589

(22) Filed: Aug. 18, 2003

Related U.S. Application Data
(60) Provisional application No. 60/404,205, filed on Aug. 16, 2002.

(51) Int. Cl. A61K 39/395; C07K 16/46

(52) U.S. Cl. 424/133.1; 424/178.1; 530/391.1

(57) ABSTRACT

Fusion proteins comprising a Receptor for Advanced Glycation End Products Ligand Binding Element (RAGE-LBE) and an immunoglobulin element are disclosed. Also disclosed are fusion proteins comprising a RAGE-LBE and a dimerization domain. Also disclosed are nucleic acids encoding such fusion proteins and methods for using disclosed nucleic acids and proteins to, for example, treat RAGE-related disorders. Additional compositions and methods are also disclosed.

### RELATED APPLICATION

This application claims the benefit of the filing date of U.S. Provisional Application No. 60/404,205, filed Aug. 16, 2002 and entitled “Methods and Compositions for Treating Rage Associated Diseases”. The entire teachings of the referenced provisional application are incorporated herein by reference.

### BACKGROUND

A number of significant human disorders are associated with an increased production of ligands for the Receptor for Advanced Glycation End Products (RAGE ligands) or increased production of RAGE itself. Consistently effective therapeutics are not available for many of these disorders, including, for example, many cancers, chronic inflammatory diseases, diabetes, amyloidoses, and cardiovascular diseases. It would be beneficial to have treatments for RAGE-related disorders.

### BRIEF SUMMARY

In certain aspects, this application relates to a fusion protein comprising a Receptor for Advanced Glycation End Product Ligand Binding Element (RAGE-LBE) and an immunoglobulin element. In certain embodiments, the RAGE-LBE comprises extracellular portions of RAGE. In certain aspects, the RAGE-LBE comprises amino acid residues 1-344, 1-330, 1-321, 1-230, or 1-118 of the amino acid sequence set forth in FIG. 7. In further embodiments, the fusion proteins of the application comprise a RAGE-LBE which comprises Ig1, Ig2, and Ig3 domains; Ig1 and Ig2 domains; or the Ig1 domain of the amino acid sequence set forth in FIG. 7. In additional embodiments, RAGE-LBE comprises one or more point mutations wherein said point mutations increase the binding affinity of said RAGE-LBE for a Receptor for Advanced Glycation End Product Binding Partner (RAGE-BP).

In certain aspects, the application relates to a fusion protein comprising a RAGE-LBE and an immunoglobulin element, wherein the immunoglobulin element comprises an immunoglobulin heavy chain. In certain embodiments, the immunoglobulin element comprises an Fc domain. In certain instances, the immunoglobulin heavy chain is selected from the group consisting of an IgM, IgD, IgE, and IgA heavy chains. In further aspects, the immunoglobulin heavy chain is selected from the group consisting of an IgG1, IgG2β, IgG2α, and IgG3 heavy chains. The immunoglobulin element may comprise the CH1 and Fc domains in certain embodiments. In certain instances, the immunoglobulin element comprises a CH1 domain of a first immunoglobulin class and a CH1 domain of a second immunoglobulin class, wherein the first and second immunoglobulin classes are not the same.

In additional embodiments, the present application relates to a fusion protein comprising a RAGE-LBE and an immunoglobulin element, further comprising a dimerizing polypeptide.

In certain embodiments, the application also relates to a composition comprising a fusion protein of the invention and a pharmaceutically acceptable carrier.

The application additionally relates to a fusion protein comprising a RAGE-LBE and a second domain selected from the group consisting of a dimerizing polypeptide, a purification polypeptide, a stabilizing polypeptide, and a targeting polypeptide. In certain embodiments, the dimerizing polypeptide comprises an amphiphilic polypeptide. The amphiphilic polypeptide may comprise up to 50 amino acids, up to 30 amino acids, up to 20 amino acids, or up to 10 amino acids. In certain embodiments, the dimerizing polypeptide comprises a peptide helix bundle. In certain embodiments, the dimerizing polypeptide comprises a leucine zipper. The leucine zipper may be a jun zipper or a fos zipper. In certain embodiments, the dimerizing polypeptide comprises a polypeptide having positively or negatively charged residues wherein said polypeptide binds to another peptide bearing opposite charges.

In further embodiments, the application relates to a fusion protein comprising an amino acid sequence that is at least 90% identical to the amino acid sequence of FIG. 3A. In certain aspects, the application relates to a nucleic acid sequence encoding a polypeptide fusion comprising a RAGE-LBE and an immunoglobulin element. In certain embodiments, the application relates to a nucleic acid sequence encoding a polypeptide at least 90% identical to the amino acid sequence set forth in FIG. 3A. In certain embodiments, the nucleic acid sequence encodes a RAGE-LBE that is fused to an immunoglobulin element through the C— or N-terminal amino or carboxy groups. The RAGE-LBE may comprise extracellular portions of RAGE. In certain embodiments, the nucleic acid sequences of the application encode a RAGE-LBE, which comprises amino acid residues 1-344, 1-330, 1-321, 1-230, or 1-118 of the amino acid sequence set forth in FIG. 7. In further embodiments, the RAGE-LBE comprises Ig1, Ig2, and Ig3 domains; Ig1 and Ig2 domains; or an Ig1 domain. In additional embodiments, the application relates to a nucleic acid sequence encoding a RAGE-LBE polypeptide comprising one or more point mutations wherein said point mutations increase the binding affinity of said RAGE-LBE for a RAGE-BP. In certain embodiments, the nucleic acid sequence encodes a RAGE-LBE that comprises one or more point mutations wherein said point mutations increase the binding affinity of said RAGE-LBE for a RAGE-BP.

In certain aspects, the application relates to a nucleic acid sequence encoding a fusion protein comprising a R AGE-LBE and an immunoglobulin element, wherein the immunoglobulin element comprises an immunoglobulin heavy chain. In certain embodiments, the immunoglobulin element comprises an Fc domain. In certain instances, the immunoglobulin heavy chain is selected from the group consisting of an IgM, IgD, IgE, and IgA heavy chains. In further aspects, the immunoglobulin heavy chain is selected from the group consisting of an IgG1, IgG2β, IgG2α, and IgG3 heavy chains. The immunoglobulin element may comprise the CH1 and Fc domains in certain embodiments. In certain instances, the immunoglobulin element comprises a CH1 domain of a first immunoglobulin class and a CH1 domain of a second immunoglobulin class, wherein the first and second immunoglobulin classes are not the same.

In additional embodiments, the present application relates to a nucleic acid sequence encoding a fusion protein comprising a RAGE-LBE and an immunoglobulin element, further comprising a second domain selected from the group consisting of a dimerizing polypeptide, a stabilizing polypeptide, a purification polypeptide, and a targeting polypeptide.

In a further embodiment, the nucleic acids of the application further comprise a transcriptional regulatory sequence operably linked to the nucleotide sequence so as to render the nucleic acid suitable for use as an expression vector. In certain embodiments, the nucleic acid further comprises a promoter wherein said promoter enhances expression of the nucleic acid molecule in mammalian cells. The application additionally relates to an expression vector comprising a nucleic acid of the present application. In certain embodiments, the expression vector replicates in at least one of a prokaryotic cell and a eukaryotic cell. The application further relates to a host cell transfected with an expression vector of the present application. Additionally, the application provides a method of producing a RAGE-LBE-Immunoglobulin fusion protein comprising culturing a host cell of the application in a cell culture medium suitable for expression of the fusion protein, and optionally, the method further comprises a purification procedure to increase the purity of said fusion protein.

In certain embodiments, the application provides an isolated antibody, or fragment thereof, specifically immunoreactive with an epitope of the amino acid sequence as set forth in FIG. 3A. In certain embodiments, the antibody is specifically immunoreactive with an epitope of amino acid residues 1-330, 1-321, 1-230, or 1-118 of the amino acid sequence as set forth in FIG. 7. In certain embodiments, the antibody inhibits binding of RAGE to one or more RAGE-BPs. In certain embodiments, the application provides an isolated antibody, or fragment thereof, specifically immunoreactive with an epitope of the amino acid sequence as set forth in FIG. 3A, wherein said antibody is selected from the group consisting of a polyclonal antibody, a monoclonal antibody, an Fab fragment, and a single chain antibody. Optionally, the antibody is labeled with a detectable label. The application additionally relates to a purified preparation of polyclonal antibody of the present application.

In a further embodiment, the application relates to a protein complex comprising one or more fusion proteins, wherein said fusion proteins are selected from the group consisting of: a) a fusion protein comprising a RAGE-LBE and an immunoglobulin element; and b) a fusion protein comprising a RAGE-LBE and a second domain selected from the group consisting of a dimerizing domain, a stabilizing domain, a purification domain, and a targeting domain.

The application additionally relates to a pharmaceutical composition comprising a RAGE-LBE and a TNF-α inhibitor. In certain embodiments, the application relates to a pharmaceutical composition comprising a fusion protein and a TNF-α inhibitor, wherein said fusion protein comprises a RAGE-LBE and an immunoglobulin element. The application further relates to a pharmaceutical composition comprising a fusion protein, wherein said fusion protein comprises a RAGE-LBE and an immunoglobulin element. In certain aspects, the RAGE-LBE comprises extracellular portions of RAGE. In certain embodiments, the RAGE-LBE comprises amino acid residues 1-344, 1-330, 1-321, 1-230, or 1-118 of the amino acid sequence as set forth in FIG. 7. In certain embodiments, the RAGE-LBE comprises Ig1, Ig2, and Ig3 domains; Ig1 and Ig2 domains; or an Ig1 domain.

In certain aspects, the RAGE-LBE of the pharmaceutical compositions of the present application comprises one or more point mutations wherein said point mutations increase the binding affinity of said RAGE-LBE for a RAGE-BP. In certain embodiments, the pharmaceutical compositions of the present application comprise a TNF-α inhibitor, wherein the TNF-α inhibitor is selected from the group consisting of a small molecule, an antibody, a peptidomimetic, and a TNFRII-Fc fusion protein.

In certain embodiments, the pharmaceutical compositions of the present application comprise an immunoglobulin element, wherein the immunoglobulin element comprises an immunoglobulin heavy chain. In certain embodiments, the immunoglobulin element comprises an Fc domain. In certain instances, the immunoglobulin heavy chain is selected from the group consisting of an IgM, IgD, IgE, and IgA heavy chains. In further aspects, the immunoglobulin heavy chain is selected from the group consisting of an IgG1, IgG2β, IgG2α, and IgG3 heavy chains. The immunoglobulin element may comprise the CH1 and Fc domains in certain embodiments. In certain instances, the immunoglobulin element comprises a CH1 domain of a first immunoglobulin class and a CH1 domain of a second immunoglobulin class, wherein the first and second immunoglobulin classes are not the same. In additional embodiments, the present application relates to a pharmaceutical composition comprising a RAGE-LBE, which further comprises a dimerizing polypeptide.

In certain embodiments, the application relates to a method of identifying a compound which inhibits interaction of a RAGE-BP polypeptide selected from the group consisting of S100 and amphoterin, with a receptor polypeptide selected from the group consisting of RAGE, RAGE-LBE, and RAGE-LBE-Immunoglobulin fusion, comprising: a) forming a reaction mixture including: (i) a RAGE-BP polypeptide of S100 or amphoterin; (ii) a receptor polypeptide of RAGE, RAGE-LBE or RAGE-LBE-Immunoglobulin fusion; and (iii) a test compound, under conditions where, in the absence of the test compound, the RAGE-BP polypeptide and the receptor polypeptide interact; and b) detecting interaction of the RAGE-BP polypeptide with the receptor polypeptide, wherein a decrease in the interaction of the RAGE-BP polypeptide and the receptor polypeptide in the presence of the test compound, relative to the level of interaction in the absence of the test compound, indicates an inhibitory activity for the test compound. In certain embodiments, the RAGE-BP is S100 (such as S100B or S100a12) or amphoterin.

The application further relates to a method of identifying a compound which inhibits the RAGE signaling activity induced by a RAGE-BP polypeptide selected from the group consisting of S100 and amphoterin, comprising: a) contacting a cell with a RAGE-BP polypeptide of S100 or amphoterin; b) contacting the cell with a test compound, under conditions where, in the absence of the test compound, the signaling activity of the RAGE occurs normally; and c) detecting the signaling activity of the RAGE induced by the RAGE-BP, wherein a decrease in the signaling activity of the RAGE induced by the RAGE-BP in the presence of the test compound, relative to the level of signaling activity in the absence of the test compound, indicates an inhibitory activity for the test compound. In certain embodiments, the RAGE-BP is S100 (such as S100B or S100a12) or amphoterin. In certain aspects, a compound which inhibits the RAGE signaling activity induced by a RAGE-BP inhibits the activation of NF-kB transcriptional activity or the activation of mitogen-activated protein kinase (MAPK) activity.

In an additional embodiment, the application provides a method of inhibiting the interaction between RAGE and a RAGE-BP comprising administering a fusion protein comprising RAGE-LBE and an immunoglobulin. In an additional embodiment, the application relates to a method of inhibiting the interaction between RAGE and a RAGE-BP comprising administering an antibody, or fragment thereof, specifically immunoreactive with an epitope of the amino acid sequence set forth in FIG. 3A. The application further relates to a method of inhibiting the interaction between RAGE and a RAGE-BP comprising administering a compound identified by a method of the present application.

In certain embodiments, the application provides a method of decreasing the activity of endogenous RAGE comprising administering a fusion protein comprising RAGE-LBE and an immunoglobulin. In certain aspects, the application relates to a method of decreasing the activity of endogenous RAGE comprising administering an antibody, or fragment thereof, specifically immunoreactive with an epitope of the amino acid sequence set forth in FIG. 3A. In an additional embodiment, the application relates to a method of decreasing the activity of endogenous RAGE comprising administering a compound identified by a method of the present application.

In certain embodiments, the application relates to a method of treating a RAGE-associated disorder comprising administering a fusion protein comprising RAGE-LBE and an immunoglobulin. In certain embodiments, the application relates to a method of treating a RAGE-associated disorder comprising administering an antibody, or fragment thereof, specifically immunoreactive with an epitope of the amino acid sequence set forth in FIG. 3A. In yet another embodiment, the application relates to a method of treating a RAGE-associated disorder comprising administering a compound identified by a method of the present application. In certain aspects, a composition of the present application is administered in combination with one or more of an agent useful in the treatment of one or more of the conditions selected from the group consisting of: amyloidoses, cancers, arthritis, Crohn's disease, chronic inflammatory diseases, acute inflammatory diseases, cardiovascular diseases, diabetes, complications of diabetes, prion-related disorders, vasculitis, nephropathies, retinopathies, and neuropathies. Optionally, the agent is selected from the group consisting of: anti-inflammatory agents, antioxidants, β-blockers, antiplatelet agents, ACE inhibitors, lipid-lowering agents, anti-angiogenic agents, and chemotherapeutics. In one embodiment, the agent is methotrexate. In another embodiment, the acute inflammatory disease is sepsis. In yet another embodiment, cardiovascular disease is restenosis.

In certain aspects, the RAGE-LBE in the methods listed above comprises extracellular portions of RAGE. In certain embodiments, the RAGE-LBE comprises amino acid residues 1-344, 1-330, 1-321, 1-230, or 1-1 18 of the amino acid sequence as set forth in FIG. 7. In certain embodiments, the RAGE-LBE comprises Ig1, Ig2, and Ig3 domains; Ig1 and Ig2 domains; or the Ig1 domain. In certain embodiments, the RAGE-LBE comprises one or more point mutations wherein said point mutations increase the binding affinity of said RAGE-LBE for a RAGE-BP.

The immunoglobulin element in the method listed above, in certain embodiments, comprises an immunoglobulin heavy chain. In certain embodiments, the immunoglobulin element comprises an Fc domain. In certain aspects, the immunoglobulin heavy chain is selected from the group consisting of an IgM, IgD, IgE, and IgA heavy chains. In further aspects, the immunoglobulin heavy chain is selected from the group consisting of an IgG1, IgG2β, IgG2α, and IgG3 heavy chains. The immunoglobulin element may comprise the CH1 and Fc domains in certain embodiments. In certain instances, the immunoglobulin element comprises a CH1 domain of a first immunoglobulin class and a CH1 domain of a second immunoglobulin class, wherein the first and second immunoglobulin classes are not the same.

In an additional embodiment, the application provides a method of treating a RAGE-associated disorder comprising administering a composition comprising a TNF-α inhibitor and at least one RAGE-LBE or a fusion protein comprising RAGE-LBE and an immunoglobulin. The application additionally relates to a method of treating a RAGE-associated disorder comprising administering a composition comprising at least a fusion protein comprising RAGE-LBE and an immunoglobulin.

RAGE-associated disorders treatable by methods of the application include amyloidoses, cancers, arthritis, Crohn's disease, chronic inflammatory diseases, acute inflammatory diseases, cardiovascular diseases, diabetes, complications of diabetes, prion-related disorders, vasculitis, nephropathies, retinopathies, and neuropathies. In certain aspects, the RAGE-associated disorder is Alzheimer's disease. Chronic inflammatory diseases treatable by methods of the application include rheumatoid arthritis, osteoarthritis, irritable bowel disease, multiple sclerosis, psoriasis, lupus or any other autoimmune disease. An acute inflammatory disease treatable by methods of the application includes sepsis. Cardiovascular diseases treatable by methods of the application include atherosclerosis and restenosis.

### BRIEF DESCRIPTION OF THE DRAWINGS

FIG. 1A shows the nucleotide sequence of a murine soluble RAGE-Fc fusion protein.

FIG. 1B shows the amino acid sequence of a murine soluble RAGE-Fc fusion protein.

FIG. 2A shows the nucleotide sequences of a murine soluble TNFRII.

FIG. 2B shows the amino acid sequences of a murine soluble TNFRII.

FIG. 3A shows an amino acid sequence of a human RAGE fused to the CH2, CH3 and hinge region of a mutated IgG1 heavy chain.

FIG. 3B shows the nucleotide sequence of a human RAGE fused to the CH2, CH3 and hinge region of a mutated IgG1 heavy chain.

FIG. 4 shows the total body score of mice induced to develop CIA and treated with RAGE-LBE fusion, sTNFRII or empty vector, at various days after induction of CIA.

FIG. 5 is a schematic showing various examples of RAGE-LBE fusion proteins.

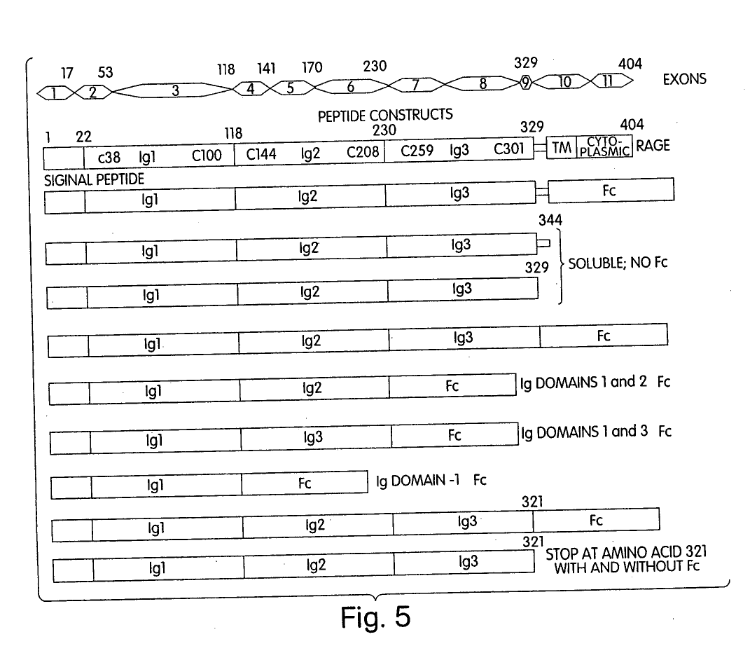

FIG. 6 shows a RAGE-LBE-Fc fusion protein binding to a RAGE ligand.

FIG. 7 shows the amino acid sequence for human RAGE.

FIG. 8 shows the nucleic acid sequence for human RAGE.

FIG. 9 shows that RAGE-LBE-Fc is secreted by CHO cells. Conditioned media was incubated overnight ± N-glycanase (to remove N-linked oligosaccharides) and subjected to SDS-PAGE (reduced). RAGE-LBE-Fc was detected with the use of antibodies specific for the Fc domain. Molecular weight shifts indicate the presence of N-linked oligosaccharides. Multiple hRAGE-LBE-Fc species suggest the possibility of additional post-translational modifications.

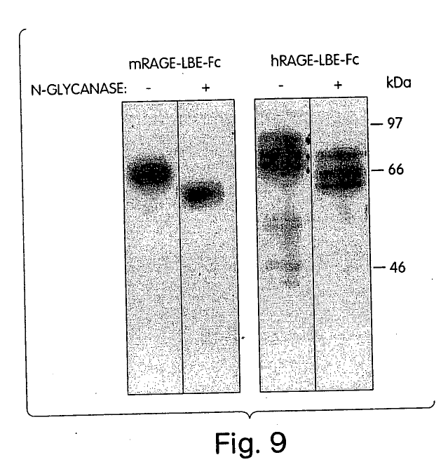

FIG. 10 shows sequence analysis of human RAGE. Analysis of human RAGE-Fc showed: 1) the N-terminal residue is glutamine (Q) which has cyclicized to form pyroglutamic acid; and 2) an N-linked modification on asparagine (N) at position two of the mature peptide.

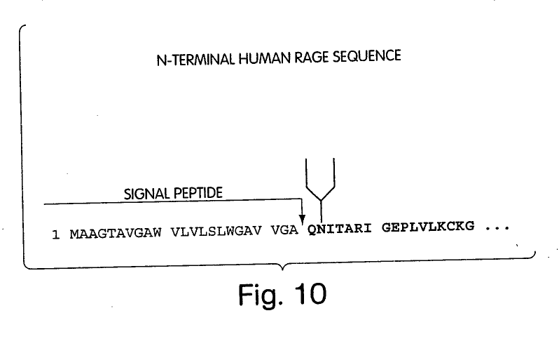

### DETAILED DESCRIPTION

### 1. DEFINITIONS

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “dimerizing polypeptide” or “dimerizing domain” includes any polypeptide that forms a dimer (or higher order complex, such as a trimer, tetramer, etc.) with another polypeptide. Optionally, the dimerizing polypeptide associates with other, identical dimerizing polypeptide, thereby forming homomultimers. An IgG Fc element is an example of a dimerizing domain that tends to form homomultimers. Optionally, the dimerizing polypeptide associates with other different dimerizing polypeptides, thereby forming heteromultimers. The Jun leucine zipper domain forms a dimer with the Fos leucine zipper domain, and is therefore an example of a dimerizing domain that tends to form heteromultimers. Dimerizing domains may form both hetero- and homomultimers.

An “expression construct” is any recombinant nucleic acid that includes an expressible nucleic acid and regulatory elements sufficient to mediate expression in a suitable host cell.

The terms “fusion protein” and “chimeric protein” are interchangeable and refer to a protein or polypeptide that has an amino acid sequence having portions corresponding to amino acid sequences from two or more proteins. The sequences from two or more proteins may be full or partial (i.e., fragments) of the proteins. Fusion proteins may also have linking regions of amino acids between the portions corresponding to those of the proteins. Such fusion proteins may be prepared by recombinant methods, wherein the corresponding nucleic acids are joined through treatment with nucleases and ligases and incorporated into an expression vector. Preparation of fusion proteins is generally understood by those having ordinary skill in the art.

The term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The term “or” is used herein to mean, and is used interchangeably with, the term “and/or,” unless context clearly indicates otherwise.

The term “percent identical” refers to sequence identity between two amino acid sequences or between two nucleotide sequences. Percent identity can be determined by comparing a position in each sequence which may be aligned for purposes of comparison. Expression as a percentage of identity refers to a function of the number of identical amino acids or nucleic acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The terms “polypeptide” and “protein” are used interchangeably herein.

A “Receptor for Advanced Glycation End Products Ligand Binding Element” or “RAGE-LBE” includes any extracellular portion of a transmembrane RAGE polypeptide (e.g., soluble RAGE) and fragments thereof that retain the ability to bind a RAGE ligand.

A “Receptor for Advanced Glycation End Products Binding Partner” or “RAGE-BP” includes any substance (e.g., polypeptide, small molecule, carbohydrate structure, etc.) that binds in a physiological setting to an extracellular portion of a RAGE protein (a receptor polypeptide such as, e.g., RAGE, RAGE-LBE, or RAGE-LBE-Immunoglobulin fusion protein).

“RAGE-related disorders” or “RAGE-associated disorders” include any disorder in which an affected cell or tissue exhibits an increase or decrease in the expression and/or activity of RAGE or one or more RAGE ligands. RAGE-related disorders also include any disorder that is treatable (i.e., one or more symptom may be eliminated or ameliorated) by a decrease in RAGE function (including, for example, administration of an agent that disrupts RAGE:RAGE-BP interactions).

The term “recombinant nucleic acid” includes any nucleic acid comprising at least two sequences which are not present together in nature. A recombinant nucleic acid may be generated in vitro, for example by using the methods of molecular biology, or in vivo, for example by insertion of a nucleic acid at a novel chromosomal location by homologous or non-homologous recombination.

The term “treating” with regard to a subject, refers to improving at least one symptom of the subject's disease or disorder. Treating can be curing the disease or condition or improving it.

The term “vector” refers to a nucleic acid molecule capable of transporting another nucleic acid to which it has been linked. One type of vector is an episome, i.e., a nucleic acid capable of extra-chromosomal replication. Another type of vector is an integrative vector that is designed to recombine with the genetic material of a host cell. Vectors may be both autonomously replicating and integrative, and the properties of a vector may differ depending on the cellular context (i.e., a vector may be autonomously replicating in one host cell type and purely integrative in another host cell type). Vectors capable of directing the expression of expressible nucleic acids to which they are operatively linked are referred to herein as “expression vectors.”

### 2. FUSION PROTEINS

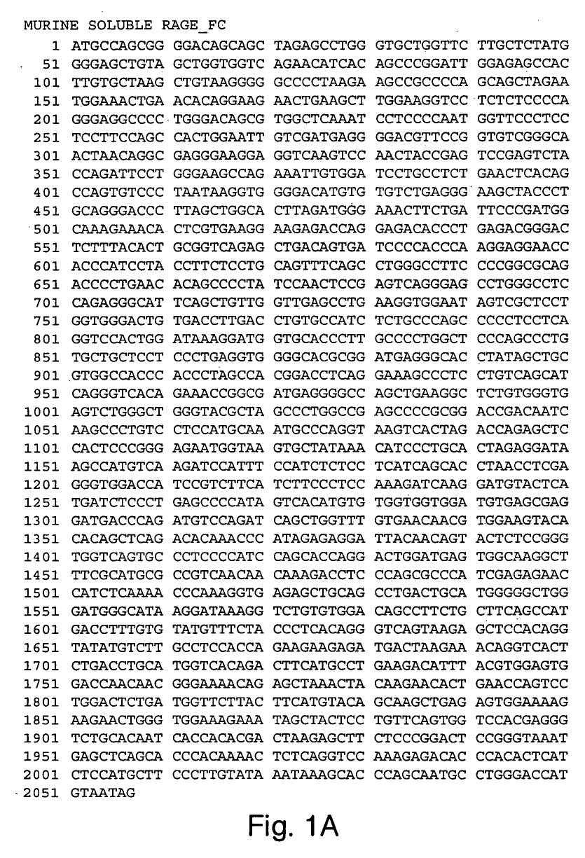

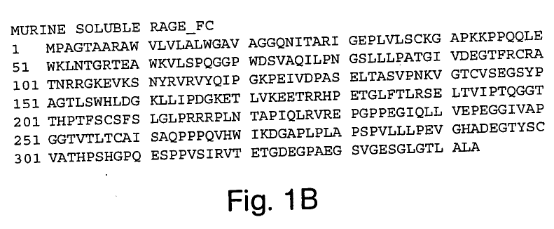

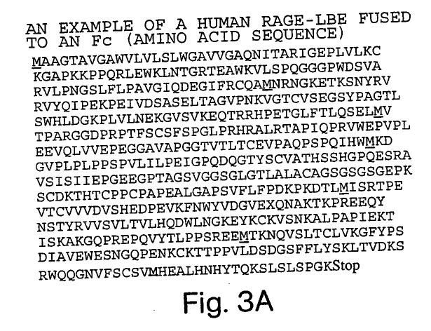

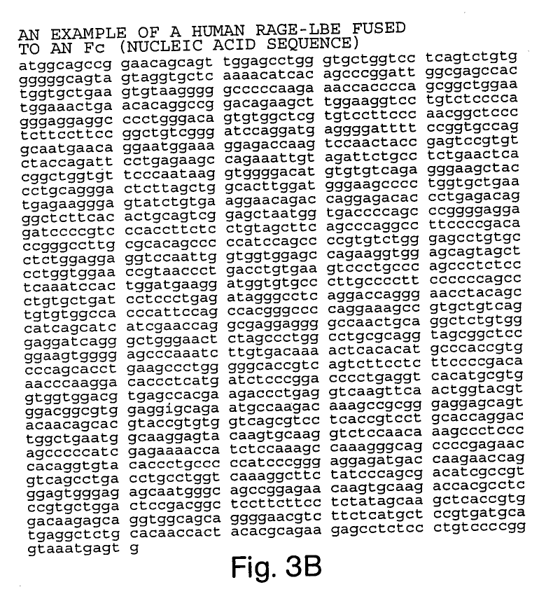

In certain aspects, fusion proteins comprising a Receptor for Advanced Glycation End Product Ligand Binding Element (RAGE-LBE) are provided. In certain embodiments, fusion proteins comprising a RAGE-LBE and an immunoglobulin element are provided (e.g., as set forth in FIG. 1B or 3A). In further embodiments, the fusion proteins comprise a RAGE-LBE and a second domain selected from the group consisting of a dimerzing domain, a targeting domain, a stabilizing domain, and a purification domain.

A RAGE-LBE may be any extracellular portion of a RAGE protein that retains the ability to bind to a RAGE ligand. In many organisms, the RAGE protein is a transmembrane protein, with a portion of the protein that is positioned inside the cell (the intracellular portion) and a portion of the protein that is positioned outside the cell (the extracellular portion). The term “RAGE ligands” is intended to encompass any substance that binds to RAGE or RAGE-LBE in a physiological setting. Exemplary RAGE ligands include nonenzymatically glycated adducts (advanced glycation endproducts), the proinflammatory cytokine-like molecules of the S100/calgranulin family, amphoterin (also known as HMG-1 or HMGB-1) and beta-sheet fibrils such as those found in amyloid structures.

In certain embodiments, the RAGE-LBE comprises a fragment of RAGE that retains an ability to bind to RAGE ligands. In certain aspects, the fusion proteins of the present invention comprise a RAGE fragment that retains an ability to bind to RAGE ligands and an immunoglobulin element. In further embodiments, the fusion proteins comprise a RAGE fragment that retains an ability to bind to RAGE ligands and a second domain selected from the group consisting of a dimerzing domain, a targeting domain, a stabilizing domain, and a purification domain.

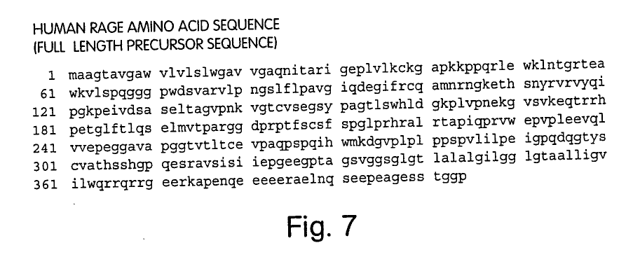

As discussed above, in certain embodiments, the RAGE-LBE comprises the extracellular portion of RAGE that retains its ability to bind to RAGE-ligands. In one aspect, the RAGE-LBE comprises Ig1, Ig2, and Ig3 domains. In another aspect, the RAGE-LBE comprises Ig1 and Ig2 domains. In other aspects, the RAGE-LBE comprises the Ig1 domain of RAGE. In yet another aspect, the RAGE-LBE comprises amino acids residues 1-344, 1-330, 1-321, 1-230, or 1-118 of the amino acid sequence as set forth in FIG. 7.

In yet another embodiment, the invention comprises amino acid sequence variants of the RAGE-LBE. These variants of RAGE-LBE are prepared keeping in mind various objectives, such as increasing the affinity of the RAGE-LBE for its ligand, facilitating the stability, purification and preparation of the binding partner, modifying its plasma half life, improving therapeutic efficacy, and lessening the severity or occurrence of side effects during therapeutic use of the composition described herein. In an illustrative embodiment, the variant RAGE-LBE fusion protein comprises an amino acid sequence that is at least 90% identical to the amino acid sequence of FIG. 3A.

Amino acid sequence variants of the RAGE-LBE fall into one or more of three classes: insertional, substitutional, or deletional variants. These variants may be prepared by methods that are well within the purview of the skilled artisan such as site-specific mutagenesis of nucleotides in the DNA encoding the RAGE-LBE, by which DNA encoding the variant is obtained, and thereafter expressing the DNA in recombinant cell culture. However, fragments having up to about 100-150 amino acid residues can be prepared conveniently by in vitro synthesis.

The amino acid sequence variants of RAGE-LBE may be predetermined variants not found in nature or may be naturally occurring alleles. The RAGE-LBE variants typically exhibit the same qualitative biological properties, for example, ligand binding activity as the naturally occurring endogenous RAGE.

While the site for introducing an amino acid sequence variation may be predetermined, the mutation per se need not be predetermined. For example, in order to optimize the performance of a mutation at a given site, random or saturation mutagenesis (where all 20 possible residues are inserted) is conducted at the target codon and the expressed RAGE-LBE variant is screened for the optimal combination of desired activities. Such screening is within the ordinary skill in the art.

Amino acid insertions usually will be on the order of about from 1 to 10 amino acid residues; substitutions are typically introduced for single residues; and deletions will range about from 1 to 30 residues. Deletions or insertions preferably are made in adjacent pairs, i.e., a deletion of 2 residues or insertion of 2 residues. It will be amply apparent from the following discussion that substitutions, deletions, insertions or any combination thereof may be introduced or combined in order to arrive at a final construct.

Insertional amino acid sequence variants of the RAGE-LBE are those in which one or more amino acid residues extraneous to the RAGE-LBE are introduced into a predetermined site in the target RAGE-LBE and which displace the preexisting residues.

Substantial changes in function may be made by selecting substitutions that are less conservative, i.e., selecting residues that differ more significantly in their effect on maintaining: (a) the structure of the polypeptide backbone in the area of the substitution, for example as a sheet or helical conformation; (b) the charge or hydrophobicity of the molecule at the target site; or (c) the bulk of the side chain. The substitutions which in general are expected to produce the greatest changes in RAGE-LBE properties will be those in which: (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine.

In general, it is reasonable to expect, for example, that an isolated replacement of a leucine with an isoleucine or valine, an aspartate with a glutamate, a threonine with a serine, or a similar replacement of an amino acid with a structurally related amino acid (i.e., conservative mutations) will not have a major effect on the biological activity of the resulting molecule. Conservative replacements are those that take place within a family of amino acids that are related in their side chains. Genetically encoded amino acids are can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. In similar fashion, the amino acid repertoire can be grouped as: (1) acidic=aspartate, glutamate; (2) basic=lysine, arginine, histidine; ( 3) aliphatic=glycine, alanine, valine, leucine, isoleucine, serine, threonine, with serine and threonine optionally be grouped separately as aliphatichydroxyl; (4) aromatic=phenylalanine, tyrosine, tryptophan; (5) amide=asparagine, glutamine; and (6) sulfur-containing=cysteine and methionine (see, for example, Biochemistry, 2nd ed., Ed. by L. Stryer, W. H. Freeman and Co., 1981). Whether a change in the amino acid sequence of a polypeptide results in a functional homolog can be readily determined by assessing the ability of the variant polypeptide to produce a response in cells in a fashion similar to the wild-type protein. For instance, such variant forms of a RAGE-LBE can be assessed, e.g., for their ability to bind to the RAGE ligands. Polypeptides in which more than one replacement has taken place can readily be tested in the same manner.

As discussed, some deletions, insertions, and substitutions will not produce radical changes in the characteristics of the RAGE-LBE molecule. However, when it is difficult to predict the exact effect of the substitution, deletion, or insertion in advance of doing so, for example when modifying an immune epitope, one skilled in the art will appreciate that the effect may be evaluated by routine screening assays. For example, a variant typically is made by site specific mutagenesis of the RAGE-LBE-encoding nucleic acid, expression of the variant nucleic acid in recombinant cell culture and, optionally, purification from the cell culture for example by immunoaffinity adsorption on a polyclonal anti-RAGE-LBE column (in order to adsorb the variant by at least one remaining immune epitope). The activity of the cell lysate or purified RAGE-LBE variant is then screened in a suitable screening assay for the desired characteristic.

Substitutional variants of the RAGE-LBE also include variants where functionally homologous domains of other proteins are substituted by routine methods for one or more of the above-identified RAGE-LBE domains. Where the variant is a fragment of a particular domain of the RAGE-LBE, it preferably but not necessarily has at least about 70% homology to the corresponding RAGE-LBE domain. Similar substitutions may desirably be made for the signal sequence, the Ig1, Ig2 or Ig³ domains.

As discussed above, the present invention provides fusion proteins comprising a RAGE-LBE and an immunoglobulin element. An immunoglobulin element may be any portion of an immunoglobulin. In certain embodiments, the immunoglobulin element comprises one or more domains of an IgG heavy chain. For example, an immunoglobulin element may comprise a heavy chain or a portion thereof from an IgG, IgD, IgA or IgM. Immunoglobulin heavy chain constant region domains include CH1, CH2, CH3, and CH4 of any class of immunoglobulin heavy chain including gamma, alpha, epsilon, mu, and delta classes. A particularly preferred immunoglobulin heavy chain constant region domain is human CH1. Immunoglobulin variable regions include VH, Vkappa or Vgamma.

In one embodiment, the RAGE-LBE is fused C-terminally to the N-terminus of the constant region of immunoglobulins in place of the variable region(s) thereof, however N-terminal fusions of the binding partner may also be constructed. Typically, such fusions retain at least functionally active hinge, CH2 and CH3 domains of the constant region of an immunoglobulin heavy chain. Fusions are also made to the C-terminus of the Fc portion of a constant domain, or immediately N-terminal to the CH1 of the heavy chain or the corresponding region of the light chain. This ordinarily is accomplished by constructing the appropriate DNA sequence and expressing it in recombinant cell culture. Alternatively, however the polypeptides of this invention may be synthesized according to known methods.

In some embodiments, the hybrid immunoglobulins are assembled as monomers, or hetero- or homo-multimers, and particularly as dimers or tetramers. Generally, these assembled immunoglobulins will have known unit structures as represented by the following diagrams. A basic four chain structural unit is the form in which IgG, IgD, and IgE exist. A four chain unit is repeated in the higher molecular weight immunoglobulins; IgM generally exists as a pentamer of basic four-chain units held together by disulfide bonds. IgA globulin, and occasionally IgG globulin, may also exist in a multimeric form in serum. In the case of multimers, each four chain unit may be the same or different.

In certain embodiments, a RAGE-LBE is fused to a dimerization domain. Dimerization domains may be essentially any polypeptide that forms a dimer (or higher order complex, such as a trimer, tetramer, etc.) with another polypeptide. Optionally, the dimerizing polypeptide associates with other, identical dimerizing polypeptides, thereby forming homomultimers. An IgG Fc element is an example of a dimerizing domain that tends to form homomultimers. Optionally, the dimerizing polypeptide associates with other different dimerizing polypeptides, thereby forming heteromultimers. The Jun leucine zipper domain forms a dimer with the Fos leucine zipper domain, and is therefore an example of a dimerizing domain that tends to form heteromultimers. Dimerizing domains may form both hetero- and homomultimers.

Different elements of fusion proteins may be arranged in any manner that is consistent with the desired functionality. For example, a RAGE-LBE may be placed C-terminal to an immunoglobulin element, or, alternatively, an immunoglobulin element may be placed C-terminal to a RAGE-LBE. The RAGE-LBE and immunoglobulin element or dimerizing polypeptide need not be adjacent in a fusion protein, and additional domains or amino acid sequences may be included C— or N-terminal to either domain or between the domains.

It will be appreciated that RAGE-LBE proteins or RAGE-LBE fusion proteins of the present invention may be modified either by natural processes such as processing and other post-translational modifications, or by chemical modification techniques which are well known in the art. Known modifications which may be present in proteins of the present invention include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Such modifications are well known to those of skill and have been described in great detail in the scientific literature. Several particularly common modifications including glycosylation, lipid attachment, sulfation, hydroxylation and ADP-ribosylation are described in most basic texts such as Proteins—Structure And Molecular Properties, 2nd Ed., T. E. Creighton, W. H. Freeman and Company, New York, 1993. Detailed reviews are also available on this subject. See, e.g., Wold, F., Posttranslational Protein Modifications: Perspectives and Prospects, pages 1-12 in Posttranslational Covalent Modification Of Proteins, B. C. Johnson, Ed., Academic Press, New York, 1983; Seifter et al., “Analysis for protein modifications and nonprotein cofactors,” Meth. Enzymol., 1990, 182:626-646 and Rattan et al, “Protein Synthesis: Posttranslational Modifications and Aging,” Ann. N.Y Acad. Sci., 1992, 663: 48-62.

### 3. NUCLEIC ACIDS

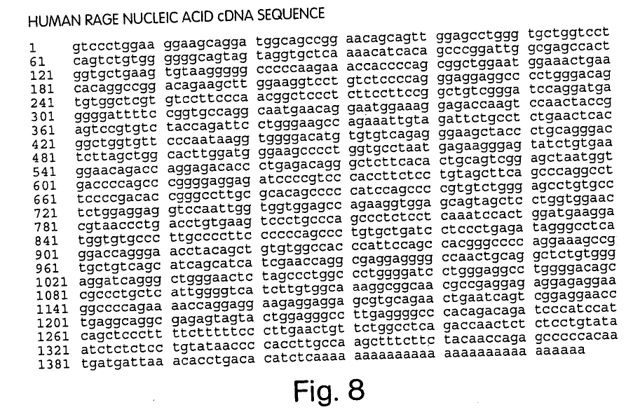

In certain aspects the invention provides nucleic acids encoding the fusion proteins disclosed herein, such as RAGE-LBE-immunoglobulin element fusion proteins and RAGE-LBE-dimerization domain fusion proteins, including all of the exemplary fusion proteins described above. In one embodiment, a nucleic acid encoding a fusion protein of the invention comprises the nucleic acid of FIG. 8 encoding human RAGE or a portion of said nucleic acid.

Nucleic acids encoding fusion proteins may also include nucleic acids that encode variants of RAGE-LBEs, immunoglobulin elements or dimerization domains (e.g., nucleic acid sequence as forth in FIG. 1A or 3B). Variant nucleotide sequences include sequences that differ by one or more nucleotide substitutions, additions or deletions, such as allelic variants; and will, therefore, include coding sequences that differ from the nucleotide sequence of RAGE, immunoglobulin or dimerization domain. Optionally, a variant will be at least 80% identical to, 90% identical to, 95% identical to, or 99% identical to the reference sequence (e.g., the sequence as set forth in FIG. 3B). Variants will also include nucleotide sequences that hybridize under stringent conditions (i.e., equivalent to about 20-27° C. below the melting temperature (Tm) of the DNA duplex formed in about 1 M salt) to the relevant reference nucleotide sequence. In an illustrative embodiment, a variant nucleic acid encodes a RAGE-LBE fusion protein comprises an amino acid sequence that is at least 90% identical to the amino acid sequence of FIG. 3A.

One of ordinary skill in the art will understand readily that appropriate stringency conditions which promote DNA hybridization can be varied. For example, one could perform the hybridization at 6.0×sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the invention provides nucleic acids which hybridize under low stringency conditions of 6×SSC at room temperature followed by a wash at 2×SSC at room temperature.

In another aspect of the invention, the subject nucleic acid is provided in an expression vector comprising a nucleotide sequence encoding a subject fusion polypeptide and operably linked to at least one regulatory sequence. Operably linked is intended to mean that the nucleotide sequence is linked to a regulatory sequence in a manner which allows expression of the nucleotide sequence. Regulatory sequences are art-recognized and are selected to direct expression of the fusion polypeptide. Accordingly, the term “regulatory sequence” includes promoters, enhancers and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding a fusion protein. Such useful expression control sequences, include, for example, the early and late promoters of SV40, tet promoter, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast a-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

As will be apparent, the subject gene constructs can be used to cause expression of the subject fusion polypeptides in cells propagated in culture, e.g., to produce proteins or polypeptides, including fusion proteins or polypeptides, for purification.

This invention also pertains to a host cell transfected with a recombinant gene including a coding sequence for one or more of the subject fusion proteins. The host cell may be any prokaryotic or eukaryotic cell, although the invention does not encompass a cell that is part of a human. For example, a polypeptide of the present invention may be expressed in bacterial cells such as E. coli, insect cells (e.g., using a baculovirus expression system), yeast, or mammalian cells. A preferred mammalian cell is a Chinese hamster ovary cell (CHO cell). Other suitable host cells are known to those skilled in the art.

Accordingly, the present invention further pertains to methods of producing the subject fusion polypeptides. For example, a host cell transfected with an expression vector encoding an fusion polypeptide can be cultured under appropriate conditions to allow expression of the polypeptide to occur. The polypeptide may be secreted and isolated from a mixture of cells and medium containing the polypeptide. Alternatively, the polypeptide may be retained cytoplasmically and the cells harvested, lysed and the protein isolated. A cell culture includes host cells, media and other byproducts. Suitable media for cell culture are well known in the art. The polypeptide can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptide. In a certain embodiments, the fusion protein contains a domain which facilitates its purification, such as a GST moiety or hexahistidine moiety. Preferably the purification portion is readily cleavable from the rest of the fusion protein.

A fusion protein of the invention can be produced by ligating the relevant cloned genes, or a portions thereof, into a vector suitable for expression in either prokaryotic cells, eukaryotic cells, or both. Expression vehicles for production of a recombinant fusion protein include plasmids and other vectors. For instance, suitable vectors for the expression of a fusion protein include plasmids of the types: pBR322-derived plasmids, pEMBL-derived plasmids, pEX-derived plasmids, pBTac-derived plasmids and pUCderived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al., (1983) in Experimental Manipulation of Gene Expression, ed. M. Inouye Academic Press, p. 83, incorporated by reference herein). These vectors can replicate in E. coli due to the presence of the pBR322 ori, and in S. cerevisiae due to the replication determinant of the yeast 2 micron plasmid. In addition, drug resistance markers such as ampicillin can be used.

The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and transformation of host organisms are well known in the art. For other suitable expression systems for both prokaryotic and eukaryotic cells, as well as general recombinant procedures, see Molecular Cloning: A Laboratory Manual, 2nd Ed., ed. by Sambrook, Fritsch and Maniatis (Cold Spring Harbor Laboratory Press, 1989) Chapters 16 and 17. In some instances, it may be desirable to express the fusion protein by the use of a baculovirus expression system. Examples of such baculovirus expression systems include pVL-derived vectors (such as pVL1392, pVL1393 and pVL9411), pAcUW-derived vectors (such as pAcUW1), and pBlueBac-derived vectors (such as the μ-gal containing pBlueBac III).

In another embodiment, a fusion gene coding for a purification leader sequence, such as a poly-(His)/enterokinase cleavage site sequence at the N-terminus of the desired portion of the recombinant protein, can allow purification of the expressed fusion protein by affinity chromatography using a Ni²⁺ metal resin. The purification leader sequence can then be subsequently removed by treatment with enterokinase to provide the purified fusion protein (e.g., see Hochuli et al., (1987) J. Chromatography 411:177; and Janknecht et al., (1991) PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

Cloned DNA sequences may be introduced into cultured mammalian cells by various methods known in the art, including electroporation, lipofection and calcium phosphate mediated transfection.

### 4. ANTIBODIES AND USES THEREFOR

Another aspect of the invention pertains to isolated antibodies specifically immunoreactive with one or more epitopes of the RAGE amino acid sequence as set forth in FIG. 3A. Preferably, the epitopes with which the antibodies are specifically immunoreactive are selected from amino acid residues 1 through 330, 1 through 321, 1 through 230, and 1 through 118 of the RAGE amino acid sequence as set forth in FIG. 7.

In certain embodiments, antibodies of the present invention are selected from a polyclonal antibody, a monoclonal antibody, an Fab fragment, and a single chain antibody. For example, anti-protein/anti-peptide antisera or monoclonal antibodies can be made by standard protocols (see, for example, Antibodies: A Laboratory Manual ed. by Harlow and Lane (Cold Spring Harbor Press: 1988)). Optionally, the antibodies are labeled with a detectable label.

In certain embodiments, antibodies of the present invention inhibit the binding of RAGE to one or more RAGE-BPs. For example, an antibody specifically immunoreactive with an epitope of amino acid residues 1-330 of the RAGE amino acid sequence of FIG. 7 can disrupt the binding of RAGE to at least one of its ligands such as advanced glycation endproducts (AGEs), amyloidogenic peptides/polypeptides, amphoterins, and S100/calgranulins.

The present invention also contemplates a purified preparation of polyclonal antibody specifically immunoreactive with one or more epitopes of the RAGE amino acid sequence as set forth in FIG. 3A.

A mammal, such as a mouse, a hamster or rabbit can be immunized with an immunogenic form of the peptide (e.g., amino acid residues 1 through 330 of RAGE amino acid sequence in FIG. 7 or an antigenic fragment which is capable of eliciting an antibody response, or a fusion protein as described above). Techniques for conferring immunogenicity on a protein or peptide include conjugation to carriers or other techniques well known in the art. An immunogenic portion of a polypeptide can be administered in the presence of adjuvant. The progress of immunization can be monitored by detection of antibody titers in plasma or serum. Standard ELISA or other immunoassays can be used with the immunogen as antigen to assess the levels of antibodies.

Following immunization of an animal with an antigenic preparation of the subject polypeptides, antisera can be obtained and, if desired, polyclonal antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with an epitope of the RAGE polypeptide and monoclonal antibodies isolated from a culture comprising such hybridoma cells.

The term “antibody” as used herein is intended to include fragments thereof which are also specifically reactive with an epitope of the RAGE polypeptide. Antibodies can be fragmented using conventional techniques and the fragments screened for utility in the same manner as described above for whole antibodies. For example, F(ab)₂ fragments c an be generated by treating antibody with pepsin. The resulting F(ab)₂ fragment can be treated to reduce disulfide bridges to produce Fab fragments.

The antibody of the present invention is further intended to include bispecific, single-chain, and chimeric and humanized molecules having affinity for one of the subject polypeptides, conferred by at least one CDR region of the antibody. In preferred embodiments, the antibody further comprises a label attached thereto and able to be detected, (e.g., the label can be a radioisotope, fluorescent compound, enzyme or enzyme co-factor).

In certain embodiments, antibodies of the present invention can be administered in combination with other agents as part of a combinatorial therapy. For example, in the case of inflammatory conditions, the subject antibodies can be administered in combination with one or more other agents useful in the treatment of inflammatory diseases or conditions. In the case of cardiovascular disease conditions, and particularly those arising from atherosclerotic plaques, which are thought to have a substantial inflammatory component, the subject antibodies can be administered in combination with one or more other agents useful in the treatment of cardiovascular diseases. In the case of cancer, the subject antibodies can be administered in combination with one or more anti-angiogenic factors, chemotherapeutics, or as an adjuvant to radiotherapy. It is further envisioned that the administration of the subject antibodies will serve as part of a cancer treatment regimen which may combine many different cancer therapeutic agents. In the case of IBD, the subject antibodies can be administered with one or more anti-inflammatory agents, and may additionally be combined with a modified dietary regimen.

Administration of the subject antibodies can be used to treat a RAGE-associated disorder, or can be used in combination with other agents and therapeutic regimens to treat a RAGE-associated disorder.

### 5. METHODS FOR INHIBITING AN INTERACTION BETWEEN A RAGE-LBE AND A RAGE-BP

Certain aspects of the invention relate to methods for inhibiting the interaction between a RAGE-LBE and a RAGE-BP. Preferably, such methods are used for treating RAGE-associated disorders.

In a preferred embodiment, such methods comprise administering a RAGE-LBE fusion protein disclosed herein. In another embodiment, such methods comprise administering an antibody, as described above, that is specifically immunoreactive with one or more epitopes of the RAGE amino acid sequence as set forth in FIG. 3A. In yet another embodiment, such methods comprise administering a compound that inhibits the binding of RAGE to one or more RAGE-BPs. Exemplary methods of identifying such compounds are discussed below in subsection 6.

In certain embodiments, the interaction is inhibited in vitro, such as in a reaction mixture comprising purified proteins, cells, biological samples, tissues, artificial tissues, etc. In certain embodiments, the interaction is inhibited in vivo, for example, by administering a RAGE-LBE fusion or causing a RAGE-LBE fusion to be produced in vivo.

In certain aspects, the invention relates to methods for treating a RAGE-related disorder by inhibiting the interaction between a RAGE-LBE and a RAGE-BP. Such methods include administering a RAGE-LBE fusion protein, an anti-RAGE antibody as described above, or an identified compound that inhibits the binding of RAGE to one or more RAGE-BPs.

### 6. METHODS FOR INHIBITING EXPRESSION OF RAGE OR RAGE-BP

Certain aspects of the present invention contemplate methods of inhibiting expression of RAGE, or a RAGE-BP (e.g., S100 or amphoterin) or both. Preferably, such methods can be used for treating RAGE-associated disorders.

In one embodiment, the invention relates to the use of antisense nucleic acid to decrease expression of RAGE or a RAGE-BP. Such an antisense nucleic acid can be delivered, for example, as an expression plasmid which, when transcribed in the cell, produces RNA which is complementary to at least a unique portion of the cellular mRNA which encodes a RAGE or a RAGE-BP polypeptide. Alternatively, the construct is an oligonucleotide which is generated ex vivo and which, when introduced into the cell causes inhibition of expression by hybridizing with the mRNA and/or genomic sequences encoding a biomarker polypeptide. Such oligonucleotide probes are optionally modified oligonucleotide which are resistant to endogenous nucleases, e.g., e xonucleases and/or endonucleases, and is therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in nucleic acid therapy have been reviewed, for example, by van der Krol et al., (1988) Biotechniques 6:958-976; and Stein et al., (1988) Cancer Res 48:2659-2668.

In another embodiment, the invention relates to the use of RNA interference (RNAi) to effect knockdown of RAGE or a RAGE-BP gene. RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. RNAi provides a useful method of inhibiting gene expression in vitro or in vivo. RNAi constructs can comprise either long stretches of dsRNA identical or substantially identical to the target nucleic acid sequence or short stretches of dsRNA identical to or substantially identical to only a region of the target nucleic acid sequence.

Optionally, the RNAi constructs contain a nucleotide sequence that hybridizes under physiologic conditions of the cell to the nucleotide sequence of at least a portion of the mRNA transcript for the gene to be inhibited (e.g., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

The subject RNAi constructs can be “small interfering RNAs” or “siRNAs.” These nucleic acids are around 19-30 nucleotides in length, and even more preferably 21-23 nucleotides in length. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group. In certain embodiments, the siRNA constructs can be generated by processing of longer double-stranded RNAs, for example, in the presence of the enzyme dicer. The combination is maintained under conditions in which the dsRNA is processed to RNA molecules of about 21 to about 23 nucleotides. The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of an nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis. Methods of chemically modifying RNA molecules can be adapted for modifying RNAi constructs (see, e.g., Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

The RNAi construct can also be in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the effects of interferon or PKR are preferred.

Alternatively, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci U S A, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

PCT application WO 01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

In another embodiment, the application relates to the use of aptamers to effect (e.g., inhibit) the activity of a RAGE polypeptide or a RAGE-BP. Aptamers are oligonucleotides that are selected to bind specifically to a desired molecular structure. Aptamers typically are RNA, but may be DNA or analogs or derivatives thereof, such as, without limitation, peptide nucleic acids (PNAs) and phosphorothioate nucleic acids. As used herein, the term “aptamer,” e.g., RNA aptamer or DNA aptamer, includes single-stranded oligonucleotides that bind specifically to a target molecule. Aptamers bind tightly and specifically to target molecules; most aptamers to proteins bind with a Kd (equilibrium dissociation constant) in the range of 1 pM to 1 nM.

Aptamers typically are the products of an affinity selection process similar to the affinity selection of phage display (also known as in vitro molecular evolution). The process involves performing several tandem iterations of affinity separation, e.g., using a solid support to which the desired immunogen is bound, followed by polymerase chain reaction (PCR) to amplify nucleic acids that bound to the immunogens. Each round of affinity separation thus enriches the nucleic acid population for molecules that successfully bind the desired immunogen. In this manner, a random pool of nucleic acids may be “educated” to yield aptamers that specifically bind target molecules. Aptamer sequences can be generated according to methods known to one of skill in the art, including, for example, the SELEX method described in the following references: U.S. Pat. Nos. 5,475,096; 5,595,877; 5,670,637; 5,696,249; 5,773,598; 5,817,785. Aptamers and methods of preparing them are also described in, for example, E. N. Brody et al. (1999) Mol. Diagn. 4:381-388, the contents of which are incorporated herein by reference.

In another embodiment, the invention relates to the use of ribozyme molecules designed to catalytically cleave an mRNA transcripts to prevent translation of mRNA (see, e.g., PCT International Publication WO 90/11364, published Oct. 4, 1990; Sarver et al., 1990, Science 247:1222-1225; and U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591. The ribozymes of the present invention also include RNA endoribonucleases (hereinafter “Cech-type ribozymes”) such as the one which occurs naturally in Tetrahymena thermophila (known as the IVS or L-19 IVS RNA) and which has been extensively described (see, e.g., Zaug, et al., 1984, Science, 224:574-578; Zaug and Cech, 1986, Science, 231:470-475; Zaug, et al., 1986, Nature, 324:429-433; published International patent application No. WO88/04300 by University Patents Inc.; Been and Cech, 1986, Cell, 47:207-216).

In a further embodiment, the invention relates to the use of DNA enzymes to inhibit expression of RAGE or a RAGE-BP gene. DNA enzymes incorporate some of the mechanistic features of both antisense and ribozyme technologies. DNA enzymes are designed so that they recognize a particular target nucleic acid sequence, much like an antisense oligonucleotide, however much like a ribozyme they are catalytic and specifically cleave the target nucleic acid. Briefly, to design an ideal DNA enzyme that specifically recognizes and cleaves a target nucleic acid, one of skill in the art must first identify the unique target sequence. Preferably, the unique or substantially sequence is a G/C rich of approximately 18 to 22 nucleotides. High G/C content helps insure a stronger interaction between the DNA enzyme and the target sequence. When synthesizing the DNA enzyme, the specific antisense recognition sequence that will target the enzyme to the message is divided so that it comprises the two arms of the DNA enzyme, and the DNA enzyme loop is placed between the two specific arms. Methods of making and administering DNA enzymes can be found, for example, in U.S. Pat. No. 6,110,462.

### 7. METHODS OF TREATMENT

Certain embodiments of the invention relate to methods of treating RAGE-related disorders. RAGE-related disorders may be characterized generally as including any disorder in which an affected cell exhibits elevated expression of RAGE or one or more RAGE ligands. RAGE-related disorders may also be characterized as any disorder that is treatable (i.e., one or more symptoms may be eliminated or ameliorated) by a decrease in RAGE function. For example, RAGE function can be decreased by administration of an agent that disrupts the interaction between RAGE and a RAGE-BP. Alternatively, RAGE function can be decreased by administration of an agent (e.g., antisense nucleic acids, or RNAi constructs) that inhibits expression of RAGE or a RAGE-BP gene as described above.

The increased expression of RAGE is associated with several pathological states, such as diabetic vasculopathy, nephropathy, retinopathy, neuropathy, and other disorders, including Alzheimer's disease and immune/inflammatory reactions of blood vessel walls. RAGE ligands are produced in tissue affected with many inflammatory disorders, including arthritis (such as rheumatoid arthritis). In diabetic tissues, the production of RAGE is thought to be caused by the overproduction of advanced glycation endproducts. This results in oxidative stress and endothelial cell dysfunction that leads to vascular disease in diabetics.

Deposition of amyloid in tissues causes a variety of toxic effects on cells and are characteristic of a suite of diseases that may be termed amyloidoses. RAGE binds to beta-sheet fibrillar material, such as that found in amyloid-beta peptide, Abeta, amylin, serum amyloid A and prion-derived peptides. RAGE is also expressed at increased levels in tissues having amyloid structures. Accordingly, RAGE is involved in amyloid disorders. The RAGE-amyloid interaction is thought to result in oxidative stress leading to neuronal degeneration.

A variety of RAGE ligands, and particularly those of the S100/calgranulin family, are produced in inflamed tissues. This observation is true both for acute inflammation, such as that seen in response to a lipopolysaccharide challenge (as in sepsis) and for chronic inflammation, such as that seen in various forms of arthritis, ulcerative colitis, inflammatory bowel disease, etc. Cardiovascular diseases, and particularly those arising from atherosclerotic plaques, are also thought to have a substantial inflammatory component. Such diseases include occlusive, thrombotic and embolic diseases, such as angina, fragile plaque disorder and embolic stroke, respectively. All of these may be considered RAGE-related disorders.

Tumor cells also evince an increased expression of a RAGE ligand, particularly amphoterin, indicating that cancers are also a RAGE-related disorder. Furthermore, the oxidative effects and other aspects of chronic inflammation may have a contributory effect to the genesis of certain tumors.

Accordingly, the list of RAGE-related disorders that may be treated with an inventive composition include: amyloidoses (such as Alzheimer's disease), arthritis, Crohn's disease, chronic inflammatory diseases (such as rheumatoid arthritis, osteoarthritis, ulcerative colitis, irritable bowel disease, multiple sclerosis, psoriasis, lupus and other autoimmune diseases), acute inflammatory diseases (such as sepsis), shock (e.g., septic shock, hemorrhagic shock), cardiovascular diseases (e.g., atherosclerosis, stroke, fragile plaque disorder, angina and restenosis), diabetes (and particularly cardiovascular diseases in diabetics), complications of diabetes, prion-related disorders, cancers, vasculitis and other vasculitis syndromes such as necrotizing vasculitides, nephropathies, retinopathies, and neuropathies.

In certain preferred embodiments, the invention relates to a method for treating an arthritis, the method comprising administering a RAGE-LBE fusion protein. Optionally, the fusion protein may be administered as a polypeptide, e.g., as part of a pharmaceutical composition. In a particularly preferred embodiment, the fusion protein may be administered by administering a nucleic acid encoding the fusion protein and designed to express the fusion protein in a cell of the subject.

In certain aspects, the present invention provides for the administration of the subject fusion proteins. The subject fusion proteins can be administered, in vitro or in vivo, and expression of the subject fusion proteins can be achieved either by administering the subject fusion proteins themselves or by administering nucleic acids encoding the subject fusion proteins. In certain embodiments, the subject fusion proteins or nucleic acids are administered as pharmaceutical compositions. In certain other embodiments, the subject fusion proteins or nucleic acids are administered with one or more additional agents. In yet another aspect of the present invention, the administration of the subject fusion proteins is part of a therapeutic regimen to treat a particular condition. Conditions which can be treated by administration of either the subject proteins/nucleic acids alone, or by administration of the subject proteins/nucleic acids in combination with other agents, include RAGE-associated disorders. By way of example, RAGE-associated disorders include, but are not limited to, rheumatoid arthritis, osteoarthritis, inflammatory bowel disease, atherosclerosis, vasculitis and other vasculitis syndromes such as necrotizing vasculitides, Alzheimer's disease, cancer, complications of diabetes such as diabetic retinopathy, autoimmune diseases such as psoriasis and lupus. RAGE-associated disorders further include acute inflammatory diseases (e.g., sepsis), chronic inflammatory diseases, and other conditions which are aggravated by inflammation (i.e., the symptoms of which may be ameliorated by decreasing inflammation).

A wide variety of methods are well known in the art for the delivery of nucleic acids encoding particular proteins (e.g., a nucleic acid encoding a subject fusion protein). Expression constructs used for in vitro or in vivo administration may be administered in any biologically effective carrier (e.g., any formulation or composition capable of effectively delivering the expression construct). Approaches include insertion of the subject gene in viral vectors which function by directly transfecting cells. Exemplary viral vectors include recombinant retroviruses, adenovirus, adeno-associated virus, herpes simplex virus-1, and lentivirus. Additional approaches include the use of recombinant bacterial or eukaryotic plasmids. Delivery of plasmid DNA can be facilitated by, for example, cationic liposomes (lipofectin) or derivatized (e.g., antibody conjugated), polylysine conjugates, gramacidin S, artificial viral envelopes or other such intracellular carriers, and CaPO₄ precipitation. In some instances, expression constructs can be delivered directly by injection to the specific cells or tissues in which expression is desired. One of skill in the art can readily select among these delivery systems depending on the cell or tissue in which expression is desired, whether administration is to be systemic or local, and the desired dose of expression.

One particular approach for administering a nucleic acid expressing a subject fusion protein (an expression construct) is by the use of a viral vector containing a nucleic acid encoding a subject fusion protein. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up the vector.

Retrovirus vectors and adeno-associated virus vectors are generally understood to be the recombinant gene delivery system of choice for the transfer of exogenous genes in vivo, particularly into humans. These vectors provide efficient delivery of genes into cells, and the transferred nucleic acids may be stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. ( 1990) Blood 76: 271). Thus, recombinant retrovirus c an be constructed in which part of the retroviral coding sequence (gag, pol, env) has been replaced by nucleic acid encoding a subject fusion protein, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include ΨCrip, ΨCre, ψ2 and ψAm. Retroviruses have been used to introduce a variety of genes into many different cell types, including epithelial cells, in vitro and/or in vivo (see for example Eglitis, et al. (1985) Science 230: 1395-1398; Danos and Mulligan (1988) Proc. Natl. Acad. Sci. USA 85: 6460-6464; Wilson et al. (1988) Proc. Natl. Acad. Sci. USA 85: 3014-3018; Armentano et al. (1990) Proc. Natl. Acad. Sci. USA 87: 6141-6145; Huberet al. (1991) Proc. Natl. Acad. Sci. USA 88: 8039-8043; Ferry et al. (1991) Proc. Natl. Acad. Sci. USA 88: 8377-8381; Chowdhury et al. (1991) Science 254: 1802-1805; van Beusechem et al. (1992) Proc. Natl. Acad. Sci. USA 89: 7640-7644; Kay et al. (1992) Human Gene Therapy 3: 641-647; Dai et al. (1992) Proc. Natl. Acad. Sci. USA 89: 10892-10895; Hwu et al. (1993) J. Immunol. 150: 4104-4115; U.S. Pat. No. 4,868,116; U.S. Pat. No. 4,980,286; PCT Publication WO 89/07136; PCT publication WO 89/02468; PCT publication WO 89/05345; and PCT publication WO 92/07573).

Furthermore, it has been shown that it is possible to limit the infection spectrum of retroviruses and consequently of retroviral-based vectors, by modifying the viral packaging proteins on the surface of the viral particle (see, for example, PCT publications WO 93/25234 and WO 94/06920). For instance, strategies for the modification of the infection spectrum of retroviral vectors include: coupling antibodies specific for cell surface antigens to the viral env protein (Roux et al. (1989) PNAS 86: 9079-9083; Julan et al. (1992) J. Gen Virol 73: 3251-3255; and Goud et al. (1983) Virology 163: 251-254); or coupling cell surface receptor ligands to the viral env proteins (Neda et al. (1991) J Biol Chem 266: 14143-14146). Coupling can be in the form of the chemical cross-linking with a protein or other variety receptor-ligand drug, as well as by generating fusion proteins (e.g., single-chain antibody/env fusion proteins).

Another viral gene delivery system useful in the present invention utilitizes adenovirus-derived vectors. The genome of an adenovirus can be manipulated such that it encodes and expresses a gene product of interest but is inactivated in terms of its ability to replicate in a normal lytic viral life cycle. See for example Berkner et al. (1988) BioTechniques 6: 616; Rosenfeld et al. (1991) Science 252: 431-434; and Rosenfeld et al. (1992) Cell 68: 143-155. Suitable adenoviral vectors derived from the adenovirus strain Ad type5 d1324 or other strains of adenovirus (e.g., Ad2, Ad3, Adz, etc.) are well known to those skilled in the art. The virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57: 267). Most replication-defective adenoviral vectors currently in use and therefore favored by the present invention are deleted for all or parts of the viral E1 and E3 genes but retain as much as 80% of the adenoviral genetic material (see, e.g., Jones et al. (1979) Cell 16: 683; Berkner et al., supra; and Graham et al. in Methods in Molecular Biology, E. J. Murray, Ed. (Humana, Clifton, N.J., 1991) vol. 7. pp. 109-127). Expression of the inserted gene can be under control of, for example, the EIA promoter, the major late promoter (MLP) and associated leader sequences, the E3 promoter, or exogenously added promoter sequences.

Yet another viral vector system useful for delivery of the subject genes and genes encoding the subject fusion proteins is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158: 97-129). It is also one of the few viruses that may integrate its DNA into non-dividing cells, and exhibits a high frequency of stable integration (see for example Flotte et al. (1992) Am. J. Respir. Cell. Mol. Biol. 7: 349-356; Samulski et al. (1989) J. Virol. 63: 3822-3828; and McLaughlin et al. (1989) J. Virol. 62: 1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5: 3251-3260 can be used to introduce the subject genes or a nucleic acid encoding the subject fusion proteins into cells. A variety of nucleic acids have been introduced into different cell types using AAV vectors (see for example Hernonat et al. (1984) Proc. Natl. Acad. Sci. USA 81: 6466-6470; Tratschin et al. (1985) Mol. Cell. Biol. 4: 2072-2081; Wondisford et al. (1988) Mol. Endocrinol. 2: 32-39; Tratschin et al. (1984) J. Virol. 51: 611-619; and Flotte et al. (1993) J. Biol. Chem. 268: 3781-3790).

Other viral vector systems that may have application in administering expression constructs have been derived from herpes virus, vaccinia virus, lentivirus, and several RNA viruses.

In addition to viral transfer methods, such as those illustrated above, non-viral methods can also be employed to administer expression constructs including bacterial and eukaryotic expression constructs. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject genes or nucleic acids encoding subject fusion proteins by the targeted cell. Exemplary gene delivery systems of this type include liposomal derived systems, poly-lysine conjugates, and artificial viral envelopes.

In a representative embodiment, a subject gene or a nucleic acid encoding a subject fusion protein can be entrapped in liposomes bearing positive charges on their surface (e.g., lipofectins) and (optionally) which are tagged with antibodies or ligands for cell surface antigens (Mizuno et al. (1992) No Shinkei Geka 20: 547-551; PCT publication W091/06309; Japanese patent application 1047381; and European patent publication EP-A-43075). In another example, the liposomes can be tagged with monoclonal antibodies specific for antigens present in joints (e.g., as for treating arthritis and other conditions of the cartilage and/or joints). Similarly, this method can be modified to specifically target the subject proteins to any tissue to more specifically treat a condition which affects that tissue (e.g., cancer of a particular tissue, IBD, rheumatoid arthritis, vasculitis, etc.)

The actual administration of any of the foregoing gene delivery systems can be by any of a number of methods, each of which is familiar in the art. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression oft he receptor gene, or a combination thereof. In other embodiments, initial delivery of the recombinant gene is more limited with introduction into the animal being quite localized. For example, the gene delivery vehicle can be introduced into the a specific tissue by catheter (see U.S. Pat. No. 5,328,470), by stereotactic injection (e.g., Chen et al. (1994) PNAS 91: 3054-3057), orby electroporation (Dev et al. ((1994) Cancer Treat Rev 20:105-115).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced in tact from recombinant cells, e.g., retroviral vectors, the pharmaceutical preparation can comprise one or more cells which produce the gene delivery system.

Methods of administration of either nucleic acid based or protein based compositions can be by any of a number of methods well known in the art. These methods include local or systemic administration and further include intradermal, intramuscular, intraperitoneal, intravenous, subcutaneous, oral, and intranasal routes of administration. In addition, it may be desirable to introduce the pharmaceutical compositions of the invention into the central nervous system by any suitable route, including intraventricular and intrathecal injection. Intraventricular injection may be facilitated by an intraventricular catheter, for example, attached to a reservoir, such as an Ommaya reservoir. Methods of introduction may also be provided by rechargeable or biodegradable devices. Furthermore, it is contemplated that administration may occur by coating a device, implant, stent, or prosthetic.

For example, cartilage severely damaged by conditions of the joints such as rheumatoid arthritis and osteoarthritis can be replaced, in whole or in part, by various prosthetics. A variety of suitable transplantable materials exist including those based on collagen-glycosaminoglycan templates (Stone et al. (1990) Clin Orthop Relat Red 252: 129), isolated chondrocytes (Grande et al. (1989) J Orthop Res 7: 208; and Takigawa et al. (1987) Bone Miner 2: 449), and chondrocytes attached to natural or synthetic polymers (Walitani et al. (1989) J Bone Jt Surg 71B: 74; Vacanti et al. (1991) Plast Reconstr Surg 88: 753; von Schroeder et al. (1991) J Biomed Mater Res 25:329; Freed et al. (1993) J Biomed Mater Res 27: 11; and the Vacanti et al. U.S. Pat. No. 5,041,138). For example, chondrocytes can be grown in culture on biodegradable, biocompatible highly porous scaffolds formed from polymers such as polyglycolic acid, polylactic acid, agarose gel, or other polymers that degrade over time as function of hydrolysis of the polymer backbone into innocuous monomers. The matrices are designed to allow adequate nutrient and gas exchange to the cells until engraftment occurs. The cells can be cultured in vitro until adequate cell volume and density has developed for the cells to be implanted. One advantage of the matrices is that they can be cast or molded into a desired shape on an individual basis, so that the final product closely resembles the patient's own ear or nose (by way of example), or flexible matrices can be used which allow for manipulation at the time of implantation, as in a joint.

These and other implants and prosthetics can be treated with the subject fusion proteins or with an expression construct containing a nucleic acid expressing a subject fusion protein. In this way, the subject fusion proteins can be administered directly to the specific affected tissue (e.g., to the damaged joint).

In another embodiment of the present invention, the subject fusion proteins or antibodies can be administered as part of a combinatorial therapy with other agents. Combination therapy refers to any form of administration in combination of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or is a separate formulation, either concomitantly or sequentially. Thus, an individual who receives such treatment can have a combined (conjoint) effect of different therapeutic compounds.

For example, in the case of inflammatory conditions, the subject proteins or antibodies can be administered in combination with one or more other agents useful in the treatment of inflammatory diseases or conditions. Agents useful in the treatment of inflammatory diseases or conditions include, but are not limited to, anti-inflammatory agents, or antiphlogistics. Antiphlogistics include, for example, glucocorticoids, such as cortisone, hydrocortisone, prednisone, prednisolone, fluorcortolone, triamcinolone, methylprednisolone, prednylidene, paramethasone, dexamethasone, betamethasone, beclomethasone, fluprednylidene, desoxymethasone, fluocinolone, flumethasone, diflucortolone, clocortolone, clobetasol and fluocortin butyl ester; immunosuppressive agents such as anti-TNF agents (e.g., etanercept, infliximab) and IL-1 inhibitors; penicillamine; non-steroidal anti-inflammatory drugs (NSAIDs) which encompass anti-inflammatory, analgesic, and antipyretic drugs such as salicyclic acid, celecoxib, difunisal and from substituted phenylacetic acid salts or 2phenylpropionic acid salts, such as alclofenac, ibufenac, ibuprofen, clindanac, fenclorac, ketoprofen, fenoprofen, indoprofen, fenclofenac, diclofenac, flurbiprofen, pirprofen, naproxen, benoxaprofen, carprofen and cicloprofen; oxicam derivatives, such as piroxicam; anthranilic acid derivatives, such as mefenamic acid, flufenamic acid, tolfenamic acid and meclofenamic acid, anilino-substituted nicotinic acid derivatives, such as the fenamates miflumic acid, clonixin and flunixin; heteroarylacetic acids wherein heteroaryl is a 2-indol-3-yl or pyrrol-2-yl group, such as indomethacin, oxmetacin, intrazol, acemetazin, cinmetacin, zomepirac, tolmetin, colpirac and tiaprofenic acid; idenylacetic acid of the sulindac type; analgesically active heteroaryloxyacetic acids, such as benzadac; phenylbutazone; etodolac; nabumetone; and disease modifying antirheumatic drugs (DMARDs) such as methotrexate, gold salts, hydroxychloroquine, sulfasalazine, ciclosporin, azathioprine, and leflunomide.

Other therapeutics useful in the treatment of inflammatory diseases or conditions include antioxidants. Antioxidants may be natural or synthetic. Antioxidants are, for example, superoxide dismutase (SOD), 21-aminosteroids/aminochromans, vitamin C or E, etc. Many other antioxidants are well known to those of skill in the art.

The subject proteins or antibodies may serve as part of a treatment regimen for an inflammatory condition, which may combine many different anti-inflammatory agents. For example, the subject fusion proteins or antibodies may be administered in combination with one or more of an NSAID, DMARD, or immunosuppressant. In one embodiment of the application, the subject fusion proteins may be administered in combination with methotrexate. In another embodiment, the subject fusion proteins may be administered in combination with a TNF-α inhibitor.

In the case of cardiovascular disease conditions, and particularly those arising from atherosclerotic plaques, which are thought to have a substantial inflammatory component, the subject proteins or antibodies can be administered in combination with one or more other agents useful in the treatment of cardiovascular diseases. Agents useful in the treatment of cardiovascular diseases include, but are not limited to, β-blockers such as carvedilol, metoprolol, bucindolol, bisoprolol, atenolol, propranolol, nadolol, timolol, pindolol, and labetalol; antiplatelet agents such as aspirin and ticlopidine; inhibitors of angiotensin-converting enzyme (ACE) such as captopril, enalapril, lisinopril, benazepril, fosinopril, quinapril, ramipril, spirapril, and moexipril; and lipid-lowering agents such as mevastatin, lovastatin, simvastatin, pravastatin, fluvastatin, atorvastatin, and rosuvastatin.

In the case of cancer, the subject proteins or antibodies can be administered in combination with one or m ore anti-angiogenic factors, chemotherapeutics, or a s an adjuvant to radiotherapy. It is further envisioned that the administration of the subject proteins or antibodies will serve as part of a cancer treatment regimen which may combine many different cancer therapeutic agents. In the case of IBD, the subject fusion proteins or antibodies can be administered with one or more anti-inflammatory agents, and may additionally be combined with a modified dietary regimen.

Administration of the subject fusion proteins (either as protein compositions or as nucleic acid compositions which encode the subject proteins) can be used to treat a RAGE-associated disorder, or can be used in combination with other agents and therapeutic regimens to treat a RAGE-associated disorder.

### 8. DRUG SCREENING ASSAYS

In certain embodiments, the present invention provides assays for identifying test compounds which inhibit the binding of a RAGE-BP (e.g., S100 or amphoterin) to a receptor polypeptide (e.g., RAGE, RAGE-LBE, or RAGE-LBE-Immunoglobulin fusion protein as described above).

In certain embodiments, the assays detect test compounds which modulate the signaling activities of the RAGE receptor induced by a RAGE-BP selected from the group consisting of S100 and amphoterin. Such signaling activities include, but are not limited to binding to other cellular components, activating enzymes such as mitogen-activated protein kinases (MAPKs), activating NF-kB transcriptional activity, and the like.

A variety of assay formats will suffice and, in light of the present disclosure, those not expressly described herein will nevertheless be comprehended by one of ordinary skill in the art. Assay formats which approximate such conditions as formation of protein complexes, enzymatic activity, and may be generated in many different forms, and include assays based on cell-free systems, e.g., purified proteins or cell lysates, as well as cell-based assays which utilize intact cells. Simple binding assays can be used to detect compounds that inhibit the interaction between a RAGE-BP (e.g., S100 or amphoterin) and a receptor polypeptide (e.g., RAGE, RAGE-LBE, or RAGE-LBE-Immunoglobulin fusion protein). Compounds to be tested can be produced, for example, by bacteria, yeast or other organisms (e.g., natural products), produced chemically (e.g., small molecules, including peptidomimetics), or produced recombinantly.

In many embodiments, a cell is manipulated after incubation with a candidate compound and assayed for signaling activities of the RAGE receptor induced by a RAGE-BP (e.g., S100 or amphoterin). In certain embodiments, bioassays for such activities may include NF-KB activity assays (e.g., NF-KB luciferase or GFP reporter gene assays).

Exemplary NF-kB luciferase or GFP reporter gene assays may be carried out as described by Shona et al. (2002) FEBS Letters. 515: 119-126. Briefly, cells are transfected with an NF-kB-luciferase reporter gene. The transfected cells are then incubated with a candidate compound. Subsequently, NF-kB-stimulated luciferase activity is measured in cells treated with the compound or without the compound. Alternatively, cells can be transfected with an NF-kB-GFP reporter gene (Stratagene). The transfected cells are then incubated with a candidate compound. Subsequently, NF-kB-stimulated gene activity are monitored by measuring GFP expression with a fluorescence/visible light microscope set-up or by FACS analysis.

In certain embodiments, the present invention provides reconstituted protein preparations including a receptor polypeptide (e.g., RAGE, RAGE-LBE, or RAGE-LBE-Immunoglobulin fusion protein), and one or more RAGE-BPs (e.g., S100 or amphoterin). Assays of the present invention include labeled in vitro protein-protein binding assays, immunoassays for protein binding, and the like. The purified protein may also be used for determination of three-dimensional crystal structure, which can be used for modeling intermolecular interactions.

In certain embodiments of the present assays, a RAGE-BP polypeptide (e.g., S100 or amphoterin) or a receptor polypeptide (e.g., RAGE) can be endogenous to the cell selected to support the assays. Alternatively, a RAGE-BP polypeptide or a receptor polypeptide (e.g., RAGE-LBE or RAGE-LBE-Immunoglobulin fusion protein) can be derived from exogenous sources. For instance, fusion proteins can be introduced into the cell by recombinant techniques (such as through the use of an expression vector), as w ell as by microinjecting the fusion protein itself or mRNA encoding the fusion protein.

In further embodiments of the assays, a complex between a RAGE-BP and a receptor polypeptide can be generated in whole cells, taking advantage of cell culture techniques to support the subject assays. For example, as described below, a complex can be constituted in a eukaryotic cell culture system, including mammalian and yeast cells. Advantages to generating the subject assays in an intact cell include the ability to detect compounds which are functional in an environment more closely approximating that which therapeutic use of the compounds would require, including the ability of the compound to gain entry into the cell. Furthermore, certain of the in vivo embodiments of the assay, such as examples given below, are amenable to high through-put analysis of candidate compounds.

In certain in vitro embodiments of the present assay, a reconstituted complex comprises a reconstituted mixture of at least semi-purified proteins. By semi-purified, it is meant that the proteins utilized in the reconstituted mixture have been previously separated from other cellular proteins. For instance, in contrast to cell lysates, proteins involved in the complex formation are present in the mixture to at least 50% purity relative to all other proteins in the mixture, and more preferably are present at 90-95% purity. In certain embodiments of the subject method, the reconstituted protein mixture is derived by mixing highly purified proteins such that the reconstituted mixture substantially lacks other proteins (such as of cellular origin) which might interfere with or otherwise alter the ability to measure the complex assembly and/or disassembly.

In certain embodiments, assaying in the presence and absence of a candidate compound, can be accomplished in any vessel suitable for containing the reactants. Examples include microtitre plates, test tubes, and micro-centrifuge tubes.

In certain embodiments, drug screening assays can be generated which detect test compounds on t he basis of their ability to interfere with assembly, stability or function of a complex between a RAGE-BP (e.g., S100 or amphoterin) and a receptor polypeptide (e.g., RAGE, RAGE-LBE, or RAGE-LBE-Immunoglobulin fusion protein). In an exemplary binding assay, the compound of interest is contacted with a mixture comprising a RAGE-LBE-Immunoglobulin fusion polypeptide and a RAGE-BP such as S100 or amphoterin. Detection and quantification of the complex provide a means for determining the compound's efficacy at inhibiting interaction between the two components of the complex. The efficacy of the compound can be assessed by generating dose response curves from data obtained using various concentrations of the test compound. Moreover, a control assay can also be performed to provide a baseline for comparison. In the control assay, the formation of complexes is quantitated in the absence of the test compound.

In certain embodiments, association between the two polypeptides in a complex (e.g., a RAGE-BP and a receptor polypeptide), may be detected by a variety of techniques, many of which are effectively described above. For instance, modulation in the formation of complexes can be quantitated using, for example, detectably labeled proteins (e.g., radiolabeled, fluorescently labeled, or enzymatically labeled), by immunoassay, or by chromatographic detection. Surface plasmon resonance systems, such as those available from Biacore International AB (Uppsala, Sweden), may also be used to detect protein-protein interaction.

In certain embodiments, one polypeptide in a complex comprising a RAGE-BP and a receptor polypeptide, can be immobilized to facilitate separation of the complex from uncomplexed forms of the other polypeptide, as well as to accommodate automation of the assay. In an illustrative embodiment, a fusion protein can be provided which adds a domain that permits the protein to be bound to an insoluble matrix. For example, a GST-RAGE-LBE-Immunoglobulin fusion protein can be adsorbed onto glutathione sepharose beads (Sigma Chemical, St. Louis, Mo.) or glutathione derivatized microtitre plates, which are then combined with a potential interacting protein (e.g., an ³⁵S-labeled S100 polypeptide), and the test compound are incubated under conditions conducive to complex formation. Following incubation, the beads are washed to remove any unbound interacting protein, and the matrix bead-bound radiolabel determined directly (e.g., beads placed in scintillant), or in the supernatant after the complexes are dissociated, e.g., when microtitre plate is used. Alternatively, after washing away unbound protein, the complexes can be dissociated from the matrix, separated by SDS-PAGE gel, and the level of interacting polypeptide found in the matrix-bound fraction quantitated from the gel using standard electrophoretic techniques.

In another embodiment, a two-hybrid assay (also referred to as an interaction trap assay) can be used for detecting the interaction of two polypeptides in the complex of RAGE-LBE and RAGE-BP (see also, U.S. Pat. No. 5,283,317; Zervos et al. (1993) Cell 72: 223-232; Madura et al. (1993) J Biol Chem 268: 12046-12054; Bartel et al. (1993) Biotechniques 14: 920-924; and Iwabuchi et al. (1993) Oncogene 8: 1693-1696), and for subsequently detecting test compounds which inhibit binding between a RAGE-LBE-Immunoglobulin fusion polypeptide and a RAGE-BP polypeptide. This assay includes providing a host cell, for example, a yeast cell (preferred), a mammalian cell or a bacterial cell type. The host cell contains a reporter gene having a binding site for the DNA-binding domain of a transcriptional activator used in the bait protein, such that the reporter gene expresses a detectable gene product when the gene is transcriptionally activated. A first chimeric gene is provided which is capable of being expressed in the host cell, and encodes a “bait” fusion protein. A second chimeric gene is also provided which is capable of being expressed in the host cell, and encodes the “fish” fusion protein. In one embodiment, both the first and the second chimeric genes are introduced into the host cell in the form of plasmids. Preferably, however, the first chimeric gene is present in a chromosome of the host cell and the second chimeric gene is introduced into the host cell as part of a plasmid.

In certain embodiments, the invention provides a two-hybrid assay to identify test compounds that inhibit the binding of a RAGE-BP polypeptide (e.g., S100 and amphoterin) and a receptor polypeptide (e.g., RAGE, RAGE-LBE, or RAGE-LBE-Immunoglobulin fusion). To illustrate, a “bait” protein comprising a receptor polypeptide and a “fish” protein comprising a RAGE-BP polypeptide (such as S100 or amphoterin), are introduced in the host cell. Cells are subjected to conditions under which the bait and fish fusion proteins are expressed in sufficient quantity for the reporter gene to be activated. The interaction of the two fusion polypeptides results in a detectable signal produced by the expression of the reporter gene. Accordingly, the level of interaction between the two fusion proteins in the presence of a test compound and in the absence of the test compound can be evaluated by detecting the level of expression of the reporter gene in each case. Various reporter constructs may be used in accord with the methods of the invention and include, for example, reporter genes which produce such detectable signals as selected from the group consisting of an enzymatic signal, a fluorescent signal, a phosphorescent signal and drug resistance.

In many drug screening programs which test libraries of compounds and natural extracts, high throughput assays are desirable in order to maximize the number of compounds surveyed in a given period of time. Assays of the present invention which are performed in cell-free systems, such as may be developed with purified or semi-purified proteins or with lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or changes in enzymatic properties of the molecular target.

In certain embodiments, a complex formation between a RAGE-BP and a receptor may be assessed by immunoprecipitation and analysis of co-immunoprecipitated proteins or affinity purification and analysis of co-purified proteins. Fluorescence Resonance Energy Transfer (FRET)-based assays may also be used to determine such complex formation. Fluorescent molecules having the proper emission and excitation spectra that are brought into close proximity with one another can exhibit FRET. The fluorescent molecules are chosen such that the emission spectrum of one of the molecules (the donor molecule) overlaps with the excitation spectrum of the other molecule (the acceptor molecule). The donor molecule is excited by light of appropriate intensity within the donor's excitation spectrum. The donor then emits the absorbed energy as fluorescent light. The fluorescent energy it produces is quenched by the acceptor molecule. FRET can be manifested as a reduction in the intensity of the fluorescent signal from the donor, reduction in the lifetime of its excited state, and/or re-emission of fluorescent light at the longer wavelengths (lower energies) characteristic of the acceptor. When the fluorescent proteins physically separate, FRET effects are diminished or eliminated (see, for example, U.S. Pat. No. 5,981,200).

The occurrence of FRET also causes the fluorescence lifetime of the donor fluorescent moiety to decrease. This change in fluorescence lifetime can be measured using a technique termed fluorescence lifetime imaging technology (FLIM) (Verveer et al. (2000) Science 290: 1567-1570; Squire et al. (1999) J. Microsc. 193: 36; Verveer et al. (2000) Biophys. J. 78: 2127). Global analysis techniques for analyzing FLIM data have been developed. These algorithms use the understanding that the donor fluorescent moiety exists in only a limited number of states each with a distinct fluorescence lifetime. Quantitative maps of each state can be generated on a pixel-by-pixel basis.

To perform FRET-based assays, a RAGE-BP polypeptide (e.g., S100 or amphoterin) and a receptor polypeptide (e.g., RAGE, RAGE-LBE, or RAGE-LBE-Immunoglobulin fusion) are both fluorescently labeled. Suitable fluorescent labels are, in view of this specification, well known in the art. Examples are provided below, but suitable fluorescent labels not specifically discussed are also available to those of skill in the art. Fluorescent labeling may be accomplished by expressing a polypeptide as a fusion protein with a fluorescent protein, for example fluorescent proteins isolated from jellyfish, corals and other coelenterates. Exemplary fluorescent proteins include the many variants of the green fluorescent protein (GFP) of Aequoria victoria. Variants may be brighter, dimmer, or have different excitation and/or emission spectra. Certain variants are altered such that they no longer appear green, and may appear blue, cyan, yellow or red (termed BFP, CFP, YFP, and RFP, respectively). Fluorescent proteins may be stably attached to polypeptides through a variety of covalent and noncovalent linkages, including, for example, peptide bonds (eg., expression as a fusion protein), chemical cross-linking and biotin-streptavidin coupling. For examples of fluorescent proteins, see U.S. Pat. Nos. 5,625,048; 5,777,079; 6,066,476; 6,124,128; Prasher et al. (1992) Gene, 111: 229-233; Heim et al. (1994) Proc. Natl. Acad. Sci., USA, 91: 12501-04; Ward et al. (1982) Photochem. Photobiol., 35: 803-808 ; Levine et al. (1982) Comp. Biochem. Physiol., 72B: 77-85; Tersikh et al. (2000) Science 290: 1585-88.

FRET-based assays may be used in cell-based assays and in cell-free assays.

FRET-based assays are amenable to high-throughput screening methods including Fluorescence Activated Cell Sorting and fluorescent scanning of microtiter arrays.

In general, where a screening assay is a binding assay (whether protein-protein binding, compound-protein binding, etc.), one or more of the molecules may be joined to a label, where the label can directly or indirectly provide a detectable signal. Various labels include radioisotopes, fluorescers, chemiluminescers, enzymes, specific binding molecules, particles, e.g., magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g., albumin, detergents, etc that are used to facilitate optimal protein-protein binding and/or reduce nonspecific or background interactions. Reagents that improve the efficiency of the assay, such as protease inhibitors, nuclease inhibitors, anti-microbial compounds, etc. may be used. The mixture of components are added in any order that provides for the requisite binding. Incubations are performed at any suitable temperature, typically between 4° C. and 40° C. Incubation periods are selected for optimum activity, but may also be optimized to facilitate rapid high-throughput screening.

In certain embodiments, the invention provides complex-independent assays that are directed to a single polypeptide of the complex, such as (a RAGE-LBE-Immunoglobulin fusion protein). Such assays comprise identifying a test compound that is a candidate inhibitor of the binding of a RAGE-BP to a receptor polypeptide (e.g., RAGE, RAGE-LBE or RAGE-LBE-Immunoglobulin fusion).

In an exemplary embodiment, a compound that binds to a receptor polypeptide may be identified by using an receptor RAGE-LBE polypeptide. In an illustrative embodiment, a fusion protein of a RAGE-LBE-Immunoglobulin can be provided which adds an additional domain that permits the protein to be bound to an insoluble matrix. For example, a RAGE-LBE-Immunoglobulin fused with a GST protein can be adsorbed onto glutathione sepharose beads (Sigma Chemical, St. Louis, Mo.) or glutathione derivatized microtitre plates, which are then combined with a potential labeled binding compound and incubated under conditions conducive to binding. Following incubation, the beads are washed to remove any unbound compound, and the matrix bead-bound label determined directly, or in the supernatant after the bound compound is dissociated.

In certain embodiments, a label can directly or indirectly provide a detectable signal. Various labels include radioisotopes, fluorescers, chemiluminescers, enzymes, specific binding molecules, particles, e.g., magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures. In certain embodiments, such methods comprise forming the mixture in vitro. In certain embodiments, such methods comprise cell-based assays by forming the mixture in vivo. In certain embodiments, the methods comprise contacting a cell that expresses a receptor polypeptide (e.g., RAGE, RAGE-LBE or RAGE-LBE-Immunoglobulin fusion) or a variant thereof with the test compound.

In certain embodiments, assays are based on cell-free systems, e.g., purified proteins or cell lysates, as well as cell-based assays which utilize intact cells. Simple binding assays can be used to detect compounds that interact with the receptor polypeptide. Compounds to be tested can be produced, for example, by bacteria, yeast or other organisms (e.g., natural products), produced chemically (e.g., small molecules, including peptidomimetics), or produced recombinantly.

Optionally, test compounds identified from these assays may be used to treat RAGE-associated disorders.

### 9. PHARMACEUTICAL PREPARATIONS

The subject proteins or nucleic acids of the present invention are most preferably administered in the form of appropriate compositions. As appropriate compositions there may be cited all compositions usually employed for systemically or locally administering drugs. The pharmaceutically acceptable carrier should be substantially inert, so as not to act with the active component. Suitable inert carriers include water, alcohol, polyethylene glycol, mineral oil or petroleum gel, propylene glycol and the like. Said pharmaceutical preparations (including the subject fusion proteins or nucleic acids encoding the subject fusion proteins) may be formulated for administration in any convenient way for use in human or veterinary medicine.

Thus, another aspect of the present invention provides pharmaceutically acceptable compositions comprising an effective amount of a subject fusion protein, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam. However, in certain embodiments the subject agents may be simply dissolved or suspended in sterile water. In certain embodiments, the pharmaceutical preparation is non-pyrogenic, i.e., does not elevate the body temperature of a patient.

The phrase “effective amount” as used herein means that amount of one or more agent, material, or composition comprising one or more agents of the present invention which is effective for producing some desired effect in an animal. It is recognized that when an agent is being used to achieve a therapeutic effect, the actual dose which comprises the “effective amount” will vary depending on a number of conditions including the particular condition being treated, the severity of the disease, the size and health of the patient, the route of administration, etc. A skilled medical practitioner can readily determine the appropriate dose using methods well known in the medical arts.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (I1) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, one or more agents may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (see, for example, Berge et al. (1977) “Pharmaceutical Salts,” J. Pharm. Sci. 66:1-19).

The pharmaceutically acceptable salts of the agents include the conventional nontoxic salts or quaternary ammonium salts of the compounds, e.g., from non-toxic organic or inorganic acids. For example, such conventional nontoxic salts include those derived from inorganic acids such as hydrochloride, hydrobrornic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2acetoxybenzoic, fumaric, toluenesulfonic, methanesulfonic, ethane disulfonic, oxalic, isothionic, and the like.

In other cases, the one or more agents may contain one or more acidic functional groups and, thus, are capable of forming pharmaceutically acceptable salts with pharmaceutically acceptable bases. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (see, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred per cent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent with the carrier and, optionally, one or more accessory ingredients. In general, the formulations are prepared by uniformly and intimately bringing into association an agent of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules u sing such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such a s enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the agents.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the agents in the proper medium. Absorption enhancers can also be used to increase the flux of the agents across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of an agent, it is desirable to slow the absorption of the agent from subcutaneous or intramuscular injection. This may be accomplished by the use of a liquid suspension of crystalline or amorphous material having poor water solubility. The rate of absorption of the agent then depends upon its rate of dissolution which, in turn, may depend upon crystal size and crystalline form. Alternatively, delayed absorption of a parenterally administered agent form is accomplished by dissolving or suspending the agent in an oil vehicle.

Injectable depot forms are made by forming microencapsule matrices of the subject compounds in biodegradable polymers such as polylactide-polyglycolide. Depending on the ratio of agent to polymer, and the nature of the particular polymer employed, the rate of agent release can be controlled. Examples of other biodegradable polymers include poly(orthoesters) and poly(anhydrides). Depot injectable formulations are also prepared by entrapping the agent in liposomes or microemulsions which are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Apart from the above-described compositions, use may be made of covers, e.g., plasters, bandages, dressings, gauze pads and the like, containing an appropriate amount of a therapeutic. As described in detail above, therapeutic compositions may be administered/delivered on stents, devices, prosthetics, and implants.

Exemplification

The invention now being generally described, it will be more readily understood by reference to the following examples, which are included merely for purposes of illustration of certain aspects and embodiments of the present invention, and are not intended to limit the invention.

### EXAMPLE 1

### Identification of Genes that Are Up- or Down-Regulated in Patients Having Rheumatoid Arthritis

This Example describes the identification of several genes which are up- or downregulated in peripheral blood mononuclear cells (PBMCs) of subjects having rheumatoid arthritis (R.A.) relative to expression in PBMCs of normal subjects.

PMBCs were isolated from 9 patients with R.A. and 13 normal volunteers as follows. Eight mls of blood were drawn into a CPT Vacutainer tube which was inverted several times. The tube was centrifuged at 1500× g (2700 rpm) in a swinging bucket rotor at room temperature. The serum was removed and PBMCs were transferred to a 15 ml conical centrifuge tube. The cells were washed with the addition of phosphate buffered saline (PBS) and centrifuged at 450 g (1200 rpm) for 5 minutes. The supernatant was discarded and the wash procedure was repeated once more. After removal of the supernatant, total RNA was isolated with the use of the RNeasy minikit, (Qiagen, Hidden, Germany) according to the manufacturers procedure.

RNA was analyzed on oligonucleotide arrays composed of 6,800 and 12,000 human genes (Affymetrix Hu6800 and HgU95A chip sets, respectively), as follows.

Target nucleic acid for hybridization was prepared as follows. Total RNA was prepared for hybridization by denaturing 5 pg of total RNA from PBMC's for 10 minutes at 70° C. with 100 pM T7/T24-tagged oligo-dt primer (synthesized at Genetics Institute, Cambridge, Mass.), and cooled on ice. First strand cDNA synthesis was performed under the following buffer conditions: 1× first strand buffer (Invitrogen Life Technologies, Carlsbad, Calif.), 10 mM DTT (GIBCO/Invitrogen), 500 μM of each dNTP (Invitrogen Life Technologies), 400 units of Superscript RT II (Invitrogen Life Technologies) and 40 units RNAase inhibitor (Ambion, Austin, Tex.). The reaction proceeded at 47° C. for 1 hour. Second strand CDNA was synthesized with the addition of the following reagents at the final concentrations listed: 1× second strand buffer (invitrogen Life Technologies), an additional 200 μM of each dNTP (Invitrogen Life Technologies), 40 units of E. coli DNA polymerase I (Invitrogen Life Technologies), 2 units E. coli RNaseH (Invitrogen Life Technologies), and 10 units of E. coli DNA ligase. The reaction proceeded at 15.8° C. for 2 hours and during the last five minutes of this reaction 6 units of T4 DNA polymerase (New England Biolabs, Beverly, Mass.) was added. The resulting double stranded cDNA was purified with the use of BioMag carboxyl terminated particles as follows: 0.2 mg of BioMag particles (Polysciences Inc., Warrington, Pa.) were equilibrated by washing three times with 0.5 M EDTA and resuspended at a concentration of 22.2 mg/ml in 0.5 M EDTA. The double stranded cDNA reaction was diluted to a final concentration of 10% PEG/1.25 M NaCl and the bead suspension was added to a final bead concentration of 0.614 mg/ml. The reaction was incubated at room temperature for 10 minutes. The cDNA/bead complexes were washed with 300 μl of 70% ethanol, the ethanol was removed and the tubes were allowed to air dry. The cDNA was eluted with the addition of 20 μl of 10 mM Tris-acetate, pH 7.8, incubated for 2-5 minutes and the cDNA containing supernatant was removed.

10 μl of purified double stranded cDNA was then added to an in vitro transcription (IVT) solution which contained, 1× IVT buffer (Ambion, Austin, Tex.) 5,000 units T7 RNA polymerase (Epicentre Technologies, Madison, Wis.), 3 mM GTP, 1.5 mM ATP, 1.2 mM CTP and 1.2 mM UTP (Amersham/Pharmacia,), 0.4 mM each bio-16 UTP and bio-11 CTP (Enzo Diagnostics, Farrningdale, N.Y.), and 80 units RNase inhibitor (Ambion, Austin, Tex.). The reaction proceeded at 37° C. for 16 hours. Labeled RNA was purified with the use of an RNeasy (Qiagen). The RNA yield was quantitated by measuring absorbance at 260 nm.

Array Hybridization and Detection of Fluorescence was performed as follows. 12 μg of IVT was fragmented in 40 mM Tris-actetate, pH 8.0, 100 mM potassium acetate, and 30 mM magnesium acetate for 35 minutes at 94° C. The fragmented, labeled RNA probes were diluted in hybridization buffer at a final composition of 1× 2-NMorpholinoethanesulfonic acid (MES (buffer (pH 6.5), 50 pM Bio948 (control biotinylated oligo that hybridizes to landmark features on the probe array (Genetics Institute, Cambridge, Mass.)), 100 μg/ml herring sperm DNA (Promega, Madison, Wis.), 500 μg/ml acetylated BSA (Invitrogen Life Technologies) and 1 μl/μg standard curve reagent (Proprietary reagent supplied by Gene Logic, Gaithersburg, Md.). This hybridization solution was pre-hybridized with two glass beads (Fisher Scientific, Pittsburgh, Pa.) at 45° C. overnight. The hybridization solution was removed to a clean tube, heated for 1-2 min at 95° C. and microcentrifuged on high for 2 minutes to pellet insoluble debris. Affymetrix oligonucleotide array cartridges (human 6800 array P/N900183 and human U95A (Affymetrix, Santa Clara, Calif.)) were pre-wet with non-stringent wash buffer (0.9 M NaCl, 60 mM sodium phosphate, 6 mM EDTA and 0.01% Tween20) and incubated at 45° C. with rotation for 5-10 minutes. Buffer was removed from the Affymetrix cartridges and the arrays were hybridized with 180 μl of the hybridization solution at 45° C. rotating at 45-60 rpm overnight. After overnight incubation, the hybridization solutions were removed and the cartridges were filled with non-stringent wash buffer. The array cartridges were washed using an Affymetrix fluidics station according with 10 cycles of 2 mixes/cycle non-stringent wash buffer at 25° C. followed by 4 cycles of 15 mixes/cycle stringent wash buffer (100 mM MES, 0.1 M Na⁺, 0.01% Tween20 and 0.005% antifoam). The probe array was then first stained for 10 minutes at 25° C. in SAPE solution (100 mM MES, 1 M Na⁺, 0.05% Tween20, 0.005% antifoam, 2 mg/ml acetylated BSA (Invitrogen Life Technologies), and 10 μg/ml R phycoerythrin streptavidin (Molecular Probes, Eugene, Oreg.)). After first staining, the probe array was washed for 10 cycles of 4 mixes/cycle with non-stringent wash buffer at 25° C. The probe array was then stained for 10 minutes at 25° C. in antibody solution (100 mM MES, 1 M Na⁺, 0.05% Tween20, 0.005% antifoam, 2 mg/ml acetylated BSA (Invitrogen Life Technologies), 100 μg/ml Goat IgG (SIGMA, St. Louis, Mo.) and 3 μg/ml biotinylated anti-streptavidin antibody (goat) (Vector Laboratories). Following the second stain, the probe array is stained again for an additional 10 minutes at 25° C. in SAPS solution. Finally, the probe array is washed for 15 cycles of 4 mixes/cycle with non-stringent wash buffer at 30° C. Arrays were scanned using an Affymetrix gene chip scanner (Affymetrix, Santa Clara, Calif.). The scanner contains a scanning confocal microscope and uses an argon ion laser for the excitation source and emission is detected by a photomultiplier tube at 530 nm bandpass filter (fluorscein 0 or 560 longpass filter (phycoerythrin).

Data analysis was performed using GENECHIP 3.0 or 4.0 software with norm alizing/scaling to internal controls. For each patient, two parameters were used to filter the data: 1) “Absolute Decision,” which indicates the presence (P) or absence (A) of RNA of a gene within a given RNA sample; 2) “Frequency,” which measures the number of copies of a given RNA within a RNA sample, and this value is expressed as Copies per million transcripts. If a gene was called “Absent,” its frequency was not used to calculate the average frequency of the gene. If a gene was called “Absent” for more than four patients in the Hu6800 data; more than two patients in the HgU95A data, or more than six normals, no average frequency was calculated. Genes that had average frequencies for normal volunteers only were tagged “Normal” while those that had average frequencies for patients only were tagged “Disease.” The fold change in gene expression was calculated by dividing the average gene frequency of the patients by that of the normals. Genes selected for analysis met the following criteria: 1) a fold change greater than 1.95 or less than −1.95 and 2) those genes tagged as either “Normal” or “Disease.”

Of particular note, RAGE ligands S100a9 and S100a12a12 were overexpressed in cells of subjects with rheumatoid arthritis.

### EXAMPLE 2

### Identification of Genes Which Are Up- or Down-Regulated in an Animal Model of Rheumatoid Arthritis

This example describes the identification of several genes which are up or downregulated in mice having collagen induced arthritis (CIA) relative to normal mice. Gene expression was measured in paws of mice, PBMCs and insynovium.

CIA is an accepted animal model for rheumatoid arthritis. The disease was induced as follows in mice. Male DBA/1 (Jackson Laboratories, Bar Harbor, Me.) mice were used for all experiments. Arthritis was induced with the use of either chicken collagen type II (Sigma, St. Louis, Mo.) or bovine collagen type II (Chondrex, Redmond, Wash.). Chicken collagen was dissolved in 0.01 M acetic acid and emulsified with an equal volume of Complete Freund's adjuvant (CFA; Difco Labs, Detroit, Mich.) containing 1 mg/ml Mycobacterium tuberculosis (strain H37RA). 200 μg of chicken collagen was intradermally injected in the base of the tail on day 0. On day 21, mice were injected intraperitoneally with a PBS solution containing 100 μg of chicken collagen II. Bovine collagen type II (Chondrex, Redmond, Wash.) was dissolved in 0.1 M acetic acid and emulsified in an equal volume of CFA (Sigma) containing 1 m g/ml Mycobacterium tuberculosis (strain H37RA). 200 μg of bovine collagen was injected subcutaneously in the base of the tail on day 0. On day 21, mice were injected subcutaneously, in the base of the tail, with a solution containing 200 μg of bovine collagen in 0.1 M acetic acid that had been mixed with an equal volume of Incomplete Freund's adjuvant (Sigma). Naive animals received the same sets of injections, minus collagen. Mice were monitored at least three times a week for disease progression. Individual limbs were assigned a clinical score based on the index: 0=normal; P=prearthritic, characterized by focal erythema on the tips of digits; I=visible erythema accompanied by 1-2 swollen digits; 2=pronounced erythema, characterized by paw swelling and/or multi digit swelling; 3=massive swelling extending into ankle or wrist joint; 4=difficulty in use of limb or joint rigidity. The sum of all limb scores for any given mouse could yield a maximum total body score of 16.

At various stages of disease, animals were euthanized and tissues were harvested. In one series of examples, at least two paws from each animal were flash frozen in liquid nitrogen for RNA analyses. Frozen mouse paws were pulverized to a fine powder with the use of a mortar and pestle and liquid nitrogen. RNA was purified using the Promega RNAgents Total RNA Isolation System (Promega, Madison, Wis.). The RNA was further purified using the RNeasy minikit. The remaining paws were fixed in 10% formalin for histology.

In another series of examples, gene expression was determined in PBMCs of mice. Blood was collected via cardiac puncture into EDTA coated collection tubes. Blood samples were pooled according to similar total body scores (normal, prearthritic, scores 1, 3, 4, 5, 6, and 7-9) into a 15 ml conical tube. The blood was diluted 1:1 with PBS that contained 2 mM EDTA, and layered on an equal volume of Lympholyte-M (Cedar Lane Labs, Homby, Ontario, Canada). The mixture was centrifuged, with no brake, for 20 minutes at 1850 rpm in a Sorvall centrifuge (model RT 6000D). Cells at the interface were collected and added to a new tube. The cells were washed with the addition of 10 ml PBS, containing 2 mM EDTA, and centrifuged at 1200 rpm for 10 minutes. The wash was repeated two times. To lyse residual red cells, cell pellets were dispersed in 2 ml of cold 0.2% NaCl and incubated on ice for 45-60 seconds. Lysis was terminated with the addition of 2 ml of 1.6% NaCl and the cells were centrifuged at 1200 rpm for 10 minutes. PBMCs were resuspended in 5 ml of PBS, which contained 2 mM EDTA, and counted. Cells were centrifuged at 1200 rpm for 10 minutes, and the supernatant discarded in preparation for RNA isolation. Total RNA was isolated from the PBMCs using the RNeasy minikit (Qiagen, Hidden, Germany).

In yet another series of examples, RNA was obtained from isolated synovium of the diseased animals. The joint synovium was dissected from diseased and control animals under a dissection scope. Tissues from five or more animals with similar disease scores were pooled and RNA was isolated using the RNeasy kit (Qiagen, Hidden, Germany).

Gene expression was analyzed on the oligonucleotide arrays Affymetrix murine 11K chip set composed of 11,000 murine genes on two chips, murine 11 KsubA P/N 900188 and murine 11 KsubB P/N900189.

Labeled target nucleic acids for hybridization to the chips were prepared as described in the previous Example with 5 μg of PBMC RNA or 7 μg of RNA from paws or synovial tissue.

Data analysis was performed using GENECHIP 3.0 software with normalizing/ scaling to internal controls. Each experimental sample was compared to a time matched control in a two-file analysis. Next, the data were entered into the GeneSpring (Silicon Genetics, Redwood City, Calif.) analysis program. The data were filtered in a hierarchical fashion. First, the data were grouped according to paw scores. For each score, a list of genes that were called “Present” in all samples in a given score group and in the control was created. These lists were further refined by removing all genes that were not called either “Increasing” or “Decreasing” (defined in the program) in at least a majority of the samples in each score group. These lists were then filtered for genes that showed fold change greater than or equal to 1.95 or less than or equal to −1.95 in either all of the samples, if there were less than five samples, or in greater than 70% of the samples.

Of particular note, the Saa3 protein that is thought to be RAGE ligand was overexpressed in PBMCs from arthritic mice.

### EXAMPLE 3

### Biochemical Evaluation of Murine Soluble RAGE-Fc

(a) Biotinylation of the RA GE Ligand, S100B

S100B (Sigma, St. Louis, Mo.) was dissolved in a N-[2-hydroxyethyl]piperazine-N′-[3-propanesulfonic acid (EPPS; Sigma, St. Louis, Mo.) buffer to a final concentration of 50 μM. The EPPS buffer was composed of 25 mM EPPS, 150 mM NaCl, 2 mM CaCl₂, 2 mM MgCl₂, pH=7.5. Biotin (EZ-Link™ Sulfo-NHS-LC-biotin; Pierce, Rockford, Ill.) was added to the S100B solution, to a final concentration of 250 mM, for 30 min at room temperature. The biotinylation reaction was terminated when the solution was dialyzed against phosphate buffered saline at 4° C. with the use of a Slide-A-Lyzer™ dialysis cassette (Pierce, Rockford, Ill.) with a 3,500 Dalton molecular weight cutoff. After dialysis, the concentration of S100B protein was determined with the use of a BioRad Protein Assay (Bio-Rad, Hercules, Calif.).

(b) Preparation of Murine RAGE-LBE-Fc Protein

HeLa cells were used to express and secrete the RAGE-LBE-Fc protein into the cell medium. Cells were grown to ˜80% confluence in Dulbecco's Modified Eagle medium (DME) containing 10% fetal bovine serum (FBS). The medium was removed and replaced with DME containing 2% FBS and either Ad-RAGE-LBE-Fc or Ad-GFP at a concentration of approximately 10,000 viral particles per cell. After two hours at 37° C., additional DME, containing 10% FBS, was added to the cell monolayers for 26 hrs. Conditioned medium was collected and subjected to centrifugation to remove cellular debris. Aprotinin (17 μg/mL) was added to the conditioned medium which was then stored at ˜80° C. The concentration of RAGE-LBE-Fc in conditioned medium, determined with the use of an Fc-specific ELISA, was about 6 μg/mL.

(c) Evaluation of RAGE-LBE-Fc:S100B Binding

Biotinylated S100B protein (0.3-3 μM) was added to 200 μL of conditioned medium from HeLa cells that had been infected with either Ad-RAGE-LBE-Fc or Ad-GFP, as described above. The reaction volume was increased to 0.3 mL with the addition of EPPS buffer and allowed to incubate for 1.5 h at room temperature. Where indicated, some reactions contained either 45 μM of high mobility group-1 protein (HMG-1) or unlabeled S100B protein (Sigma, St. Louis, Mo.). The cross-linking reagent Bis[Sulfosuccinimidyl]suberate (BS³; Pierce, Rockford, Ill.) was added to a final concentration of 5 mM, where indicated, and the reactants incubated for an additional 45 min at room temperature. Cross-linking was terminated with the addition of Tris (Sigma, St. Louis, Mo.) to a final concentration of 200 mM. RAGE-LBE-Fc was precipitated from solution with the addition of Protein-A sepharose CL-4B (Pharmacia Biotech, Piscataway, N.J.) for 1.5 hr at room temperature. Sepharose pellets were washed 5×1 mL with a wash buffer composed of 50 mM Tris, 150 mM NaCl, 0.05% Tween-20, pH=7.5 (TBST). Captured proteins were released from the sepharose with the addition of 4× NuPageTM LDS sample buffer (Invitrogen, Carlsbad, Calif.) containing 200 mM dithiothreitol (Sigma, St. Louis, Mo.) and 4 M urea (Sigma, St. Louis, Mo.). Proteins were resolved by SDS-PAGE with the use of 4-12% gradient gels (NuPageTM Bis-iris; Invitrogen, Carlsbad, Calif.) and transferred to nitrocellulose with the use of a tank transfer unit (Hoefer Scientific, San Francisco, Calif.). Nitrocellulose was blocked with TBST containing 5% non-fat dry milk (NFDM) and then probed with a streptavidin-horse radish peroxidase conjugate (ImmunoPure Streptavidin-HRP; Pierce, Rockford, Ill.) at a dilution of 1:10,000 in TBST containing 5% NFDM. Streptavidin-HRP:biotin complexes were detected with the use of an enhanced chemiluminescent solution (Western Lightning, Perkin Elmer Life Sciences, Boston, Mass.).

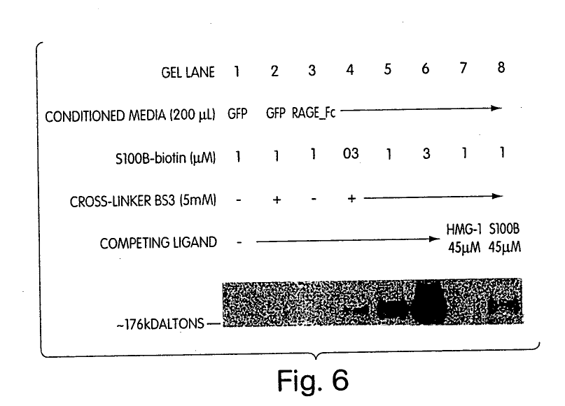

Results from a representative immunoblot are shown in FIG. 6. The band at >176 kDaltons (lanes 4-8) corresponds to biotinylated S100B cross-linked to sRAGE-Fc. This band was absent when conditioned media from cells infected with Ad-GFP (lanes 1 and 2) were evaluated. In addition, this band was absent from RAGE-LBE-Fc conditioned medium when the cross-linker BS³ was omitted (lane 3). In the presence of BS³, S100B-biotin bound RAGE-LBE-Fc in concentration-dependent manner (lanes 4-6). Moreover, this interaction was inhibited in the presence of excess HMG-1 (another RAGE ligand) and unlabeled S100B (compare lanes 7 and 8, respectively, to lane 5). Taken together, these results demonstrate that the Ad-RAGE-LBE-Fc expression vector codes for secretable RAGE-Fc that is capable of binding RAGE ligands in solution.

### EXAMPLE 4

### Identification of Cells Expressing mRAGE mRNA in Mice with CIA (Collagen Induced Arthritis)

This example describes the identity of cells expressing the mRAGE genes in mice with CIA.

Paws of mice having CIA, induced as described in Example 2, and paws of control mice were fixed for 24 hours in 4% paraformaldehyde followed by decalcification with EDTA. The tissues were trimmed, processed, embedded in paraffin, and sectioned at 5 μm for in situ hybridization. The methods for in situ hybridization were the same as those described in Example 3. Sense and antisense probes for mRAGE were prepared as follows.

Anti-sense murine RAGE and Sense murine RAGE riboprobes were produced by generating 2 independent PCR products from the corresponding transcripts. The oligonucleotides 5′-GACTGATAAT ACGACTCACT ATAGGGCGAA TGCCAGCGGG GACAGCAGCTAGAG-3′ (SEQ ID NO: 29) and 5′-AGAGGCAGGA TCCACAATTT CTGGCTTCCC AGGAAT-3′ (SEQ ID NO: 30) were used to generate a murine RAGE sense probe and 5′-GACTGATAAT ACGACTCACT ATAGGGCGAA GAGGCAGGAT CCACAATTTC TGGCTT-3′ (SEQ ID NO: 31) and 5′-ATGCCAGCGG GGACAGCAGC TAGAGCCTGG GTGCTGGTT-3′ (SEQ ID NO: 32) were used to generate a murine RAGE antisense probe.

Following PCR amplification, probes were generated using T7 RNA polyrnerase and in vitro transcription. T7 RNA polymerase binding sites were incorporated into the oligonucelotides to insert T7 binding sites at either the 5′ end of the PCR product for sense riboprobe or the 3′ end of the PCR product for antisense riboprobe. Digoxygenin labeled probes were prepared with the use of a DIG RNA labeling mix (Roche Diagnostics, Mannheim, Germany), as described by the manufacturer, and T7 RNA polymerase (Roche Diagnostics).

The probes were labeled with digoxygenin as described in Example 3. Labeled probe was detected with anti-digoxynenin antibody conjugated to horse-radish peroxidase complex (Roche) diluted 1:50 in 2% normal sheep serum/0.1% Triton X-100, for 2 hours. Labeled probe was developed with 3,3′-diaminobenzidine (Vector Laboratory, Burlingame, Calif.), for 15 minutes, washed in water, stained briefly with Mayers' hematoxylin (Sigma, St. Louis, Mo.), dehydrated through graded alcohol into xylene and mounted in DPX mountant before microscopic examination.

The results of the hybridizations are set forth in Table 1. For each tissue stained with mRAGE, the presence or the absence of a specific staining was determined. In the case of mRAGE when a specific stain was detected, the intensity of the reactivity was graded as slight, moderate and severe. A positive staining was considered to be specific when the cell(s) had a brown granular cytoplasmic staining (DAB chromogen) with an adequate reactivity of the positive and negative control sections. In this study, sense control section (negative control) was prepared for each tissue stained with mRAGE.

TABLE I
In situ hybridization evaluation of mRAGE mRNA Positive Cells
in the Control and Arthritic Paws of Mice with CIA.

| | | mRAGE mRNA |
|---|---|---|
| | mRAGE mRNA | Mouse No. 30 |
| Probe/Sample ID | Control Paw | (Arthritic Paw) |
| Macrophage | Absent | P3 |
| Lymphocyte | Absent | Negative* |
| Neutrophil | Absent | Negative |
| Mature Fibroblast | P1 | P1 |
| Immature Fibroblast | Absent | P3 |
| Activated Chondrocyte | Negative | P3 |
| Synoviocyte | Negative | Negative |
| Active Osteoblast | Negative | P3 |
| Arterial Smooth Muscle | Negative | P2 |
| Adipocyte | P1 | P2 |
| Epidermis/Follicle/Sebaceous | P2 | P3 |
| Gland | | |

P stands for cytoplasmic positivity with the various levels of positivity characterized as P1 for slightly positive, P2 for moderately positive, and P3 for severely positive.

The positive cells identified for mRAGE in the paw of mouse with induced arthritis were macrophages, activated osteoblasts, mature and immature fibroblasts, activated chondrocytes, epidermis with the follicles and sebaceous glands and the arterial smooth muscle.

The epidermis with follicles and sebaceous glands, mature fibroblasts, and adipocytes were positive in paws with and without arthritis.

### EXAMPLE 5

### Administration of RAGE-LBE Fusion Reduces CIA in Mice

This Example shows that administration of soluble RAGE to mice having CIA significantly reduces the disease, similarly to the action of soluble tumor necrosis factor receptor II (TNFRII) Fc fusion protein.

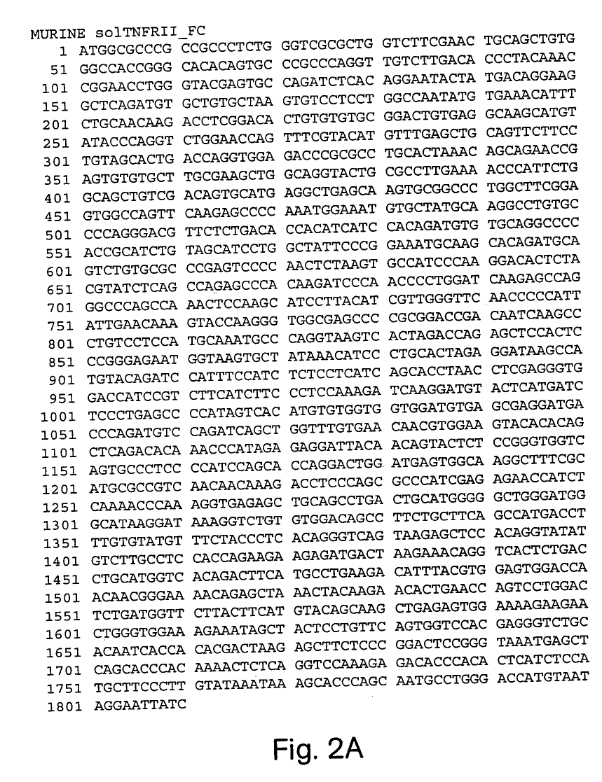

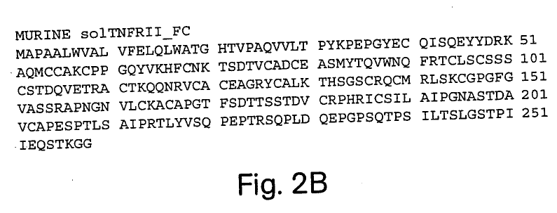

The murine RAGE was isolated from paws of DBA/1 mice with collagen induced arthritis by PCR. The coding region from the ATG at 1 to 1029 of the murine RAGE was fused to a murine IgG2a mutated Fc. The Adoril-1 mRAGE_Fc was derived by cloning the mRAGE_IG2a_Fc sequences (SEQ ID NO: 37 and encoded protein has SEQ ID NO: 38; see also FIG. 1) into EcoRI and NotI digested adenovirus vector Adori 1-2. The extracellular domain from 1-774 of the murine TNFRII was isolated from CIA diseased paws from DBA/1 mice was isolated using PCR and fused to a murine IgG2a mutated Fc. The cDNA containing the extracelular portion of mouse TNFRII fused to murine IgG2a mutated Fc (SEQ ID NO: 39 and encoded protein has SEQ ID NO: 40; see also FIG. 2) was cloned into the EcoRI and NotI of Adoril-2 and the resulting plasmid was called Adoril-2 msolTNFRII_Fc. The Adori 1-1 empty vector does not contain an insert. All constructs were verified by extensive restriction digestion analysis and sequencing of the cDNA inserts within the plasmids. Expression of all the cDNAs are driven from cytomegalovirus (CMV) immediate early promoter and enhancer.

Ad5 Ela deleted (d1327) recombinant adenovirus with or without RAGE-IgG2a_Fc or sTNFRII-IgG2a_Fc (also referred to as “sTNFRII_Fc” and “msolTNFRII_Fc”) and referred to as Ad-empty vector, Ad-RAGE_Fc and Ad-msTNFRII_Fc (vectors which were used in the CIA model) were generated by homologous recombination in a human embryonic kidney cell line 293. Recombinant adenovirus virus was isolated and subsequently amplified on 293 cells. The virus was released from infected 293 cells by three cycles of freeze thawing. The virus was further purified by two cesium chloride centrifugation gradients and dialyzed against phosphate buffered saline (PBS) pH 7.2 at 4° C. Following dialysis, glycerol was added to a concentration of 10% and the virus was stored at −80° C. until use. These viruses were characterized by expression of the transgene, plaque forming units on 293 cells, particles/ml, endotoxin measurements and PCR analysis of the virus and sequence analysis of the coding region in the virus.

The ability of the soluble mRAGE-Fc to ameliorate symptoms in a collagen-induced arthritis (CIA) murine model was examined. Male DBA/1 (Jackson Laboratories, Bar Harbor, Me.) mice were used for all experiments. Arthritis was induced with the use of bovine collagen type II (Chondrex, Redmond, Wash.). Bovine collagen type II was dissolved in 0.1 M acetic acid and emulsified in an equal volume of CFA (Sigma) containing 1 mg/ml Mycobacteriuni tuberculosis (strain H37RA). 100 μg of bovine collagen was injected subcutaneously in the base of the tail on day 0. On day 21, mice were injected subcutaneously, in the base of the tail, with a solution containing 100 μg of bovine collagen in 0.1 M acetic acid that had been mixed with an equal volume of Incomplete Freund's adjuvant (Sigma, St. Louis, Mo.). Mice received a dose of 5×10¹⁰ particles of empty virus, msolTNFRIII_Fc, or mRAGE-LBE-Fc intravenously on day 20.

Mice were monitored at least three times a week for disease progression. Individual limbs were assigned a clinical score based on the index: 0=normal; P=prearthritic, characterized by focal erythema on the tips of digits; 1=visible erythema accompanied by 1-2 swollen digits; 2=pronounced erythema, characterized by paw swelling and/or multi-digit swelling; 3=massive swelling extending into ankle or wrist joint; 4=difficulty in use of limb or joint rigidity. Thus, the sum of all limb scores for any given mouse yielded a maximum total body score of 16.

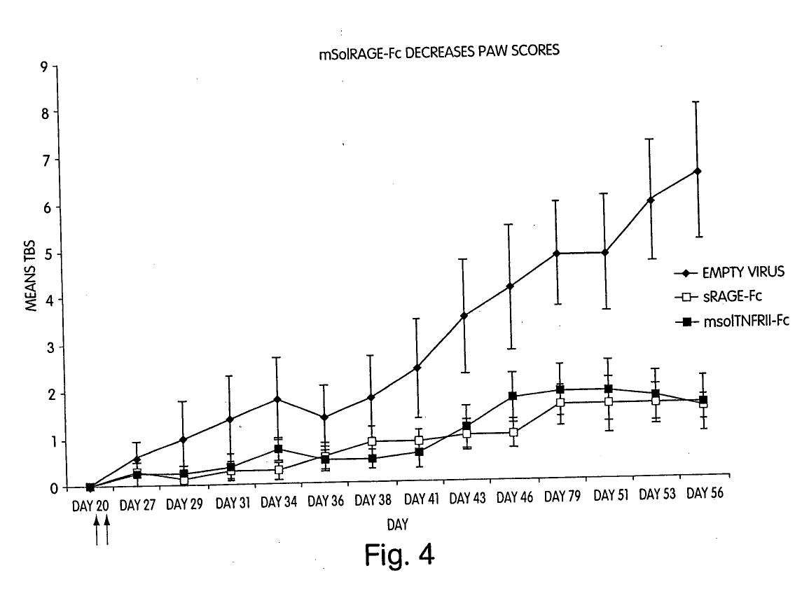

The results, which are set forth in FIG. 4, show that administration of RAGE-LBE fusion keeps the total body scores very low, indicating that administration of RAGE-LBE fusion significantly reduces and prevents CIA.

### EXAMPLE 6

### Cell Lines Which Stably Express and Secrete RAGE-LBE-FC Proteins

Stably transfected Chinese Hamster Ovary (CHO) cells were engineered to express murine and human RAGE-LBE-Fc proteins. The murine and human RAGE-LBE-FC were cloned into the mammalian expression vector, linearized and transfected into CHO cells using lipofectin (methods (Kaufman, R. J. (1990) Methods in Enzymology 185:537-66; Kaufman, R. J. (1990) Methods in Enzymology 185:487-511;Pittman, D. D. et al.(1993), Methods in Enzymology 222: 236). Cells were further selected in 20 nM and 50 nM methotrexate and conditioned medium was harvested from individual clones and analyzed by SDS sodium dodecyl sulfate-polyacrylamide gel electrophoresis (SDS-PAGE) and Western blotting to confirm expression.

CHO cells expressing the human and murine RAGE-LBE-Fc were cultured to harvest conditioned medium for protein purification. The protein was purified using standard methods. Purified protein was subjected to reducing and non-reducing SDS-PAGE, and the protein was visualized by Coomassie Blue staining (Current Protocols in Protein Sciences Wiley Interscience). The resultant analysis showed that the purified proteins were of the expected molecular weight.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A fusion protein comprising a Receptor for Advanced Glycation End Product Ligand Binding Element (RAGE-LBE) and an immunoglobulin element.

2. The fusion protein of claim 1, wherein said RAGE-LBE comprises extracellular portions of RAGE.

3. The fusion protein of claim 1, wherein said RAGE-LBE comprises amino acid residues 1 through 344 of the amino acid sequence set forth in FIG. 7.

4. The fusion protein of claim I, wherein said RAGE-LBE comprises amino acid residues 1 through 330 of the amino acid sequence set forth in FIG. 7.

5. The fusion protein of claim 1, wherein said RAGE-LBE comprises amino acid residues 1 through 321 of the amino acid sequence set forth in FIG. 7.

6. The fusion protein of claim 1, wherein said RAGE-LBE comprises amino acid residues 1 through 230 of the amino acid sequence set forth in FIG. 7.

7. The fusion protein of claim 1, wherein said RAGE-LBE comprises amino acid residues 1 through 118 of the amino acid sequence set forth in FIG. 7.

8. The fusion protein of claim 1, wherein said RAGE-LBE comprises Ig1, Ig2, and Ig3 domains.

9. The Lusion protein of claim 1, wherein said RAGE-LBE comprises Ig1 and Ig2 domains.

10. The fusion protein of claim 1, wherein said RAGE-LBE comprises the Ig1 domain.

11. The fusion protein of claim 1, wherein said RAGE-LBE comprises one or more point mutations wherein said point mutations increase the binding affinity of said RAGE-LBE for a Receptor for Advanced Glycation End Product Binding Partner (RAGE-BP).

12. The fusion protein of claim 1, wherein said immunoglobulin element comprises an immunoglobulin heavy chain.

13. The fusion protein of claim 1, wherein said immunoglobulin element comprises an Fc domain.

14. The fusion protein of claim 12, wherein said immunoglobulin heavy chain is selected from the group consisting of an IgM, IgD, IgE, and IgA heavy chains.

15. The fusion protein of claim 12, wherein said immunoglobulin heavy chain is selected from the group consisting of an IgG 1, IgG2β, IgG2α, and IgG3 heavy chains.

16. The fusion protein of claim 1, wherein said immunoglobulin element comprises the CH1 and Fc domains.

17. The fusion protein of claim 1, wherein said immunoglobulin element comprises a CH1 domain of a first immunoglobulin class and a CH1 domain of a second immunoglobulin class, wherein the first and second immunoglobulin classes are not the same.

18. The fusion protein of claim 1, further comprising a dimerizing polypeptide.

19. A composition comprising the fusion protein of any one of claims 1 to 18 and a pharmaceutically acceptable carrier.

20. A fusion protein comprising a RAGE-LBE and a second domain selected from the group consisting of a dimerizing polypeptide, a purification polypeptide, a stabilizing polypeptide, and a targeting polypeptide.

21. The fusion protein of claim 20, wherein said dimerizing polypeptide comprises an amphiphilic polypeptide.

22. The fusion protein of claim 21, wherein said amphiphilic polypeptide comprises up to 50 amino acids.

23. The fusion protein of claim 22, wherein said amphiphilic polypeptide comprises up to 30 amino acids.

24. The fusion protein of claim 22 wherein said amphiphilic polypeptide comprises up to 20 amino acids.

25. The fusion protein of claim 22, wherein said amphiphilic polypeptide comprises up to 10 amino acids.

26. The fusion protein of claim 20, wherein said dimerizing polypeptide comprises a peptide helix bundle.

27. The fusion protein of claim 20, wherein said dimerizing polypeptide comprises a leucine zipper.

28. The fusion protein of claim 27, wherein said leucine zipper is a jun zipper.

29. The fusion protein of claim 27, wherein said leucine zipper is a fos zipper.

30. The fusion protein of claim 20, wherein said dimerizing polypeptide comprises a polypeptide having positively or negatively charged residues wherein said polypeptide binds to another peptide bearing opposite charges.

31. A composition comprising the fusion protein of any one of claims 20 to 30 and a pharmaceutically acceptable carrier.

32. A fusion protein comprising an amino acid sequence that is at least 90% identical to the amino acid sequence of FIG. 3A.

33. A nucleic acid sequence encoding a polypeptide fusion comprising a RAGE-LBE and an immunoglobulin element.

34. A nucleic acid sequence encoding a polypeptide at least 90% identical to the amino acid sequence set forth in FIG. 3A.

35. The nucleic acid of claim 33, wherein said RAGE-LBE is fused to said immunoglobulin element through the C— or N-terminal amino or carboxy groups.

36. An expression vector comprising a nucleic acid of claim 33.

37. The expression vector of claim 36, which replicates in at least one of a prokaryotic cell and a eukaryotic cell.

38. A host cell transfected with the expression vector of claim 37.

39. A method of producing a RAGE-LBE-Immunoglobulin fusion protein comprising culturing the cell of claim 38 in a cell culture medium suitable for expression of the fusion protein.

40. The method of claim 39, further comprising a purification procedure to increase the purity of said fusion protein.

41. An isolated antibody, or fragment thereof, specifically immunoreactive with an epitope of the amino acid sequence as set forth in FIG. 3A.

42. A protein complex comprising one or more fusion proteins, wherein said fusion proteins are selected from the group consisting of:
a) a fusion protein comprising a RAGE-LBE and an immunoglobulin element; and
b) a fusion protein comprising a RAGE-LBE and a second domain selected from the group consisting of a dimerizing domain, a stabilizing domain, a purification domain, and a targeting domain.

43. A pharmaceutical composition comprising a RAGE-LBE and a TNF-α inhibitor.

44. A pharmaceutical composition comprising a fusion protein and a TNF-α inhibitor, wherein said fusion protein comprises a RAGE-LBE and an immunoglobulin element.

45. A method of identifying a compound which inhibits interaction of a RAGE-BP polypeptide selected from the group consisting of S100 and amphoterin, with a receptor polypeptide selected from the group consisting of RAGE, RAGE-LBE, and RAGE-LBE-Immunoglobulin fusion, comprising:
a) forming a reaction mixture including: (i) a RAGE-BP polypeptide of S100 or amphoterin; (ii) a receptor polypeptide of RAGE, RAGE-LBE or RAGE-LBE-Immunoglobulin fusion; and (iii) a test compound, under conditions where, in the absence of the test compound, the RAGE-BP polypeptide and the receptor polypeptide interact; and
b) detecting interaction of the RAGE-BP polypeptide with the receptor polypeptide,
wherein a decrease in the interaction of the RAGE-BP polypeptide and the receptor polypeptide in the presence of the test compound, relative to the level of interaction in the absence of the test compound, indicates an inhibitory activity for the test compound.

46. The method of claim 45, wherein the RAGE-BP is S100.

47. The method of claim 45, wherein the RAGE-BP is amphoterin.

48. A method of identifying a compound which inhibits the RAGE signaling activity induced by a RAGE-BP polypeptide selected from the group consisting of S100 and amphoterin, comprising:
a) contacting a cell with a RAGE-BP polypeptide of S100 or amphoterin;
b) contacting the cell with a test compound, under conditions where, in the absence of the test compound, the signaling activity of the RAGE occurs normally; and
c) detecting the signaling activity of the RAGE induced by the RAGE-BP, wherein a decrease in the signaling activity of the RAGE induced by the RAGE-BP in the presence of the test compound, relative to the level of signaling activity in the absence of the test compound, indicates an inhibitory activity for the test compound.

49. The method of claim 48, wherein the RAGE-BP is S100.

50. The method of claim 48, wherein the RAGE-BP is amphoterin.

51. The method of claim 48, wherein the signaling activity is activating NF-kB transcriptional activity.

52. The method of claim 48, wherein the signaling activity is activating mitogen-activated protein kinase (MAPK) activity.

53. A method of inhibiting the interaction between Receptor for Advanced Glycation End Product (RAGE) and a RAGE binding partner (RAGE-BP) comprising administering a fusion protein comprising RAGE-LBE and an immunoglobulin.

54. A method of inhibiting the interaction between Receptor for Advanced Glycation End Product (RAGE) and a RAGE binding partner (RAGE-BP) comprising administering the antibody of claim 41.

55. A method of inhibiting the interaction between Receptor for Advanced Glycation End Product (RAGE) and a RAGE binding partner (RAGE-BP) comprising administering a compound identified by the method of claim 45 or 48.

56. A method of decreasing the activity of endogenous RAGE comprising administering a fusion protein comprising RAGE-LBE and an immunoglobulin.

57. A method of decreasing the activity of endogenous RAGE comprising administering the antibody of claim 41.

58. A method of decreasing the activity of endogenous RAGE comprising administering a compound identified by the method of claim 45 or 48.

59. A method of treating a RAGE-associated disorder comprising administering a fusion protein comprising RAGE-LBE and an immunoglobulin.

60. The method of claim 59, wherein the fusion protein comprising RAGE-LBE and an immunoglobulin is administered in combination with one or more of an agent useful in the treatment of one or more of the conditions selected from the group consisting of: amyloidoses, cancers, arthritis, Crohn's disease, chronic inflammatory diseases, acute inflammatory diseases, cardiovascular diseases, diabetes, complications of diabetes, prion-related disorders, vasculitis, nephropathies, retinopathies, and neuropathies.

61. A method of treating a RAGE-associated disorder comprising administering the antibody of claim 41.

62. The method of claim 61, wherein said antibody is administered in combination with one or more of an agent useful in the treatment of one or more of the conditions selected from the group consisting of: amyloidoses, cancers, arthritis, Crohn's disease, chronic inflammatory diseases, acute inflammatory diseases, cardiovascular diseases, diabetes, complications of diabetes, prion-related disorders, vasculitis, nephropathies, retinopathies, and neuropathies.

63. A method of treating a RAGE-associated disorder comprising administering a compound identified by the method of claim 45 or 48.

64. The method of claim 63, wherein said compound is administered in combination with one or more of an agent useful in the treatment of one or more of the conditions selected from the group consisting of: amyloidoses, cancers, arthritis, Crohn's disease, chronic inflammatory diseases, acute inflammatory diseases, cardiovascular diseases, diabetes, complications of diabetes, prion-related disorders, vasculitis, nephropathies, retinopathies, and neuropathies.

65. The method of any one of claims 60, 62, and 64, wherein the agent is selected from the group consisting of: anti-inflammatory agents, antioxidants, β-blockers, antiplatelet agents, ACE inhibitors, lipid-lowering agents, anti-angiogenic agents, and chemotherapeutics.

66. The method of any one of claims 60, 62, and 64, wherein the agent is methotrexate.

67. The method of any one of claims 60, 62, and 64, wherein the acute inflammatory disease is sepsis.

68. The method of any one of claims 60, 62, and 64, wherein the cardiovascular disease is restenosis.

69. The method of any one of claims 53, 56, and 59, wherein said RAGE-LBE comprises extracellular portions of RAGE.

70. A method of treating a RAGE-associated disorder comprising administering a composition comprising TNF-α inhibitor and at least one RAGE-LBE or a fusion protein comprising RAGE-LBE and an immunoglobulin.

71. A method of treating a RAGE-associated disorder comprising administering a composition comprising at least a fusion protein comprising RAGE-LBE and an immunoglobulin.

72. The method of any of claims 59-64 and 70-71, wherein said RAGE-associated disorder is selected from the group consisting of amyloidoses, cancers, arthritis, Crohn's disease, chronic inflammatory diseases, acute inflammatory diseases, cardiovascular diseases, diabetes, complications of diabetes, prion-related disorders, vasculitis, nephropathies, retinopathies, and neuropathies.

73. The method of claim 72, wherein the RAGE-associated disorder is Alzheimer's disease.

74. The method of claim 72, wherein the chronic inflammatory disease is rheumatoid arthritis.

75. The method of claim 72, wherein the chronic inflammatory disease is osteoarthritis.

76. The method of claim 72, wherein the chronic inflammatory disease is irritable bowel disease.

77. The method of claim 72, wherein the chronic inflammatory disease is multiple sclerosis.

78. The method of claim 72, wherein the chronic inflammatory disease is psoriasis.

79. The method of claim 72, wherein the chronic inflammatory disease is lupus or any other autoimmune disease.

80. The method of claim 72, wherein the acute inflammatory disease is sepsis.

81. The method of claim 72, wherein the cardiovascular disease is atherosclerosis.

82. The method of claim 72, wherein the cardiovascular disease is restenosis.

83. The method of any one of claims 46 or 49 wherein S100 is S100B.

84. The method of any one of claims 46 or 49 wherein S100 is S100a12.


SEQUENCE LISTING (13 sequences)

SEQ ID NO: 1 - DNA, 2057 bp, Murine Soluble RAGE_FC
atgccagcggggacagcagctagagcctgggtgctggttcttgctctatggggagctgtagctggtggtcagaacatcacagcccggattggagagccacttgtgctaagctgtaagggggcccctaagaagccgccccagcagctagaatggaaactgaacacaggaagaactgaagcttggaaggtcctctctccccagggaggcccctgggacagcgtggctcaaatcctccccaatggttccctcctccttccagccactggaattgtcgatgaggggacgttccggtgtcgggcaactaacaggcgagggaaggaggtcaagtccaactaccgagtccgagtctaccagattcctgggaagccagaaattgtggatcctgcctctgaactcacagccagtgtccctaataaggtggggacatgtgtgtctgagggaagctaccctgcagggacccttagctggcacttagatgggaaacttctgattcccgatggcaaagaaacactcgtgaaggaagagaccaggagacaccctgagacgggactctttacactgcggtcagagctgacagtgatccccacccaaggaggaaccacccatcctaccttctcctgcagtttcagcctgggccttccccggcgcagacccctgaacacagcccctatccaactccgagtcagggagcctgggcctccagagggcattcagctgttggttgagcctgaaggtggaatagtcgctcctggtgggactgtgaccttgacctgtgccatctctgcccagccccctcctcaggtccactggataaaggatggtgcacccttgcccctggctcccagccctgtgctgctcctccctgaggtggggcacgcggatgagggcacctatagctgcgtggccacccaccctagccacggacctcaggaaagccctcctgtcagcatcagggtcacagaaaccggcgatgaggggccagctgaaggctctgtgggtgagtctgggctgggtacgctagccctggccgagccccgcggaccgacaatcaagccctgtcctccatgcaaatgcccaggtaagtcactagaccagagctccactcccgggagaatggtaagtgctataaacatccctgcactagaggataagccatgtcaagatccatttccatctctcctcatcagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggctttcgcatgcgccgtcaacaacaaagacctcccagcgcccatcgagagaaccatctcaaaacccaaaggtgagagctgcagcctgactgcatgggggctgggatgggcataaggataaaggtctgtgtggacagccttctgcttcagccatgacctttgtgtatgtttctaccctcacagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgagctcagcacccacaaaactctcaggtccaaagagacacccacactcatctccatgcttcccttgtataaataaagcacccagcaatgcctgggaccatgtaatag

SEQ ID NO: 2 - protein, 343 aa, Murine Soluble RAGE_FC
MPAGTAARAWVLVLALWGAVAGGQNITARIGEPLVLSCKGAPKKPPQQLEWKLNTGRTEAWKVLSPQGGPWDSVAQILPNGSLLLPATGIVDEGTFRCRATNRRGKEVKSNYRVRVYQIPGKPEIVDPASELTASVPNKVGTCVSEGSYPAGTLSWHLDGKLLIPDGKETLVKEETRRHPETGLFTLRSELTVIPTQGGTTHPTFSCSFSLGLPRRRPLNTAPIQLRVREPGPPEGIQLLVEPEGGIVAPGGTVTLTCAISAQPPPQVHWIKDGAPLPLAPSPVLLLPEVGHADEGTYSCVATHPSHGPQESPPVSIRVTETGDEGPAEGSVGESGLGTLALA

SEQ ID NO: 3 - DNA, 1810 bp, Murine solTNFRII_FC
atggcgcccgccgccctctgggtcgcgctggtcttcgaactgcagctgtgggccaccgggcacacagtgcccgcccaggttgtcttgacaccctacaaaccggaacctgggtacgagtgccagatctcacaggaatactatgacaggaaggctcagatgtgctgtgctaagtgtcctcctggccaatatgtgaaacatttctgcaacaagacctcggacactgtgtgtgcggactgtgaggcaagcatgtatacccaggtctggaaccagtttcgtacatgtttgagctgcagttcttcctgtagcactgaccaggtggagacccgcgcctgcactaaacagcagaaccgagtgtgtgcttgcgaagctggcaggtactgcgccttgaaaacccattctggcagctgtcgacagtgcatgaggctgagcaagtgcggccctggcttcggagtggccagttcaagagccccaaatggaaatgtgctatgcaaggcctgtgccccagggacgttctctgacaccacatcatccacagatgtgtgcaggccccaccgcatctgtagcatcctggctattcccggaaatgcaagcacagatgcagtctgtgcgcccgagtccccaactctaagtgccatcccaaggacactctacgtatctcagccagagcccacaagatcccaacccctggatcaagagccagggcccagccaaactccaagcatccttacatcgttgggttcaacccccattattgaacaaagtaccaagggtggcgagccccgcggaccgacaatcaagccctgtcctccatgcaaatgcccaggtaagtcactagaccagagctccactcccgggagaatggtaagtgctataaacatccctgcactagaggataagccatgtacagatccatttccatctctcctcatcagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggctttcgcatgcgccgtcaacaacaaagacctcccagcgcccatcgagagaaccatctcaaaacccaaaggtgagagctgcagcctgactgcatgggggctgggatgggcataaggataaaggtctgtgtggacagccttctgcttcagccatgacctttgtgtatgtttctaccctcacagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgagctcagcacccacaaaactctcaggtccaaagagacacccacactcatctccatgcttcccttgtataaataaagcacccagcaatgcctgggaccatgtaataggaattatc

SEQ ID NO: 4 - protein, 258 aa, Murine solTNFRII_FC
MAPAALWVALVFELQLWATGHTVPAQVVLTPYKPEPGYECQISQEYYDRKAQMCCAKCPPGQYVKHFCNKTSDTVCADCEASMYTQVWNQFRTCLSCSSSCSTDQVETRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGG

SEQ ID NO: 5 - protein, 585 aa, Human RAGE-LBE fused to an Fc element
  misc_feature (423)..(423): Xaa can be any naturally occurring amino acid
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQANINRNGKETKSNYRVRVYQIPEKPEIVDSASELTAGVPNKVGTCVSEGSYPAGTLSWHLDGKPLVLNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALACAGSGSGSGEPKSCDKTHTCPPCPAPEALGAPSVFLFPDKPKDTLMISRTPEVTCVVVDVSHEDPEVKFNWYVDGVEXQNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENKCKTTPPVLDSDGSFFLYSKLTDKSRWQQGNVFSCSVMHEALHNHYTQKSLSLSPGK

SEQ ID NO: 6 - DNA, 1701 bp, Human RAGE-LBE fused to an Fc element
  misc_feature (1205)..(1205): n is inosine
atggcagccggaacagcagttggagcctgggtgctggtcctcagtctgtggggggcagtagtaggtgctcaaaacatcacagcccggattggcgagccactggtgctgaagtgtaagggggcccccaagaaaccaccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgagaagccagaaattgtagattctgcctctgaactcacggctggtgttcccaataaggtggggacatgtgtgtcagagggaagctaccctgcagggactcttagctggcacttggatgggaagcccctggtgctgaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggctctgtgggaggatcagggctgggaactctagccctggcctgcgcaggtagcggctccggaagtggggagcccaaatcttgtgacaaaactcacacatgcccaccgtgcccagcacctgaagccctgggggcaccgtcagtcttcctcttccccgacaaacccaaggacaccctcatgatctcccggacccctgaggtcacatgcgtggtggtggacgtgagccacgaagaccctgaggtcaagttcaactggtacgtggacggcgtggaggngcagaatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaagtgcaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgagtg

SEQ ID NO: 7 - protein, 404 aa, Human RAGE
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETHSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 8 - DNA, 1436 bp, HUMAN RAGE
gtccctggaaggaagcaggatggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggctctgtgggaggatcagggctgggaactctagccctggccctggggatcctgggaggcctggggacagccgccctgctcattggggtcatcttgtggcaaaggcggcaacgccgaggagaggagaggaaggccccagaaaaccaggaggaagaggaggagcgtgcagaactgaatcagtcggaggaacctgaggcaggcgagagtagtactggagggccttgaggggcccacagacagatcccatccatcagctcccttttctttttcccttgaactgttctggcctcagaccaactctctcctgtataatctctctcctgtataaccccaccttgccaagctttcttctacaaccagagcccccacaatgatgattaaacacctgacacatctcaaaaaaaaaaaaaaaaaaaaaaaaaaaaaa

SEQ ID NO: 9 - protein, 40 aa, N-Terminal Human RAGE Sequence
MAAGTAVGAWVLVLSLWGAVVGAQNITARIGEPLVLKCKG

SEQ ID NO: 10 - DNA, 54 bp, primer
gactgataatacgactcactatagggcgaatgccagcggggacagcagctagag

SEQ ID NO: 11 - DNA, 36 bp, primer
agaggcaggatccacaatttctggcttcccaggaat

SEQ ID NO: 12 - DNA, 56 bp, primer
gactgataatacgactcactatagggcgaagaggcaggatccacaatttctggctt

SEQ ID NO: 13 - DNA, 39 bp, primer
atgccagcggggacagcagctagagcctgggtgctggtt